(19) United States
(12) Patent Application Publication
(10) Pub. No.: US 2024/0261429 A1
(43) Pub. Date: Aug. 8, 2024

(54) CPMV BINDING PEPTIDE

(71) Applicant: The Regents of the University of California, Oakland, CA (US)

(72) Inventors: Nicole Steinmetz, La Jolla, CA (US); Soo Khim Chan, La Jolla, CA (US)

(21) Appl. No.: 18/569,998

(22) PCT Filed: Jun. 15, 2022
(86) PCT No.: PCT/US2022/033690

§ 371 Date: Dec. 13, 2023

Related U.S. Application Data
(60) Provisional application No. 63/212,018, filed on Jun. 17, 2021.

(51) Int. Cl. A61K 47/69; A61K 47/62; C07K 7/06; C07K 14/005

(52) U.S. Cl. CPC A61K 47/6901; A61K 47/62; C07K 7/06; C07K 14/005; C07K 2319/00; C12N 2770/18022; C12N 2770/18023

(57) ABSTRACT

This disclosure provides a conjugate comprising a 7-mer peptide bound to the surface of a viral particle such as cowpea mosaic virus (CPMV). The c terminus of this peptide can be conjugated to desired molecules with the N terminus of the peptide interacting with CPMV surface.

### CROSS-REFERENCE TO RELATED APPLICATIONS

This application claims priority under 35 U.S.C. § 119(e) to U.S. Provisional Application No. 63/212,018, filed Jun. 17, 2021, the contents of which are incorporated herein by reference in their entireties.

### STATEMENT OF GOVERNMENT SUPPORT

This invention was made with government support under CA224605 awarded by the National Institutes of Health (NIH). The government has certain rights in the invention.

### BACKGROUND

The following discussion of the background is merely provided to aid the reader in the understanding the disclosure and is not admitted to describe or constitute prior art to the present disclosure. Throughout and within this disclosure, various patent and technical publications are referenced by an identifying citation or an Arabic number, the full bibliographic citation for which can be found immediately preceding the claims. These disclosure are incorporated herein to more fully describe the state of the art to which this disclosure pertains.

Cowpea mosaic virus (CPMV) is a member of the family Secoviridae. The bipartite positive sense RNA plant virus nanoparticle forms ˜30 nm-sized pseudo-T3 icosahedral proteinaceous shells comprised of 60 copies each of a large (L, 42-kDa) and small (S, 24-kDa) coat proteins.^{1,2} Ever since the discovery of CPMV in West Africa in the late 1950s,³ this nanoscale scaffold has been studied extensively and made impactful progress in many aspects of nanomedicine research. CPMV based nanotechnology has been applied in immunotherapy, vaccines, imaging and drug delivery.^{4,5} Aside from acting as an adjuvant in immunotherapy,^{6,7} CPMV capsids can be engineered to carry active ingredients such as peptides,⁸ drugs,⁹ fluorophores,¹⁰ and contrast agents.⁹ In fact, CPMV was the first plant virus developed as peptide display system^{11,12} owing to its biocompatibility and high degree of thermal and structural stability.⁹

### SUMMARY OF THE DISCLOSURE

As disclosed herein, Applicant has discovered peptides that bind to the surface of CPMV or the CMPV coat protein (CP). The C terminus of this peptide can be conjugated to desired molecules with the N terminus of the peptide interacting with CPMV or CP surface. In one aspect, an extra amino acid (lysine) is added to the C terminus of the peptide for chemical modification including biotin, fluorophore, or other chemical molecules. This CPMV-binding peptide is used as transporter/carrier to attach desired molecules onto the CPMV surface to perform a desired activity. To the best of Applicant's knowledge, CPMV-binding peptides have never been reported, highlighting the novelty of this work.

In one aspect, active ingredients are loaded and conjugated to the exterior or interior surface via covalent coupling to the coat proteins; cargo can also be infused into the nanoparticle by making use of the porous nature of the viral capsid and the chemically distinct interior/exterior environmnets.^{10,13,14} In addition, a variety of chemical bioconjugation tools can be used to modify CPMV; i.e. targeting solvent-exposed lysine side chains using N-hydroxysuccinimidyl ester (NHS) reaction and biorthogonal click-chemistry such as copper-catalyzed azide-alkyne cycloaddition (CuAAC).¹⁵ The interior can be selectively modified targeting cysteine side chains on the interior capsid surface;¹⁶ with nucleic acid-free virus-like particles of CPMV being more reactive toward this coupling strategy.¹⁷ As an alternative, genetic fusion of biological cargo can be achieved by targeting surface loops;^{11,18} the βB-βC loop on the small coat protein is most commonly used for insertion because it is highly accessible.¹⁹

In another aspect, Applicant discloses herein a non-covalent yet CPMV-specific modification strategy. In one aspect, CPMV binding peptides were isolated via phage display technology.²⁰ The peptides against CPMV are isolated through repetitive cycles of affinity selections (i.e., biopanning). The direct linkage between the genotype (gene encoding the displayed moiety) and phenotype (displayed moiety) allows the bound moieties that displayed on phage to be identified via DNA sequencing.²¹ A commercial M13 phage display 7-mer peptide library was used to pan for and select peptides binding to CPMV. The isolated CPMV binding peptides (termed CBP) were validated and selectivity, specificity, and affinity determined using a combination of western blot, native agarose gel electrophoresis, enzyme-linked immunosorbent assay (ELISA), and immunogold staining followed by transmission electron microscopy (TEM). Toward applications, Applicant also demonstrated that CPMV can be functionalized using dual-functional peptides containing the CBP domain and a receptor-targeting domain, here human epidermal growth factor receptor 2 (HER2) was targeted using a peptide ligand. Cancer cell vs. immune cell uptake of the CBP-functionalized CPMV carrying the HER2 ligand was monitored using flow cytometry to assay for targeted vs. non-specific cell uptake.

Also provided is a method to treat cancer in a subject in need thereof by administering to the subject an effective amount of the CPMV or CP conjugates further comprising an ant-cancer agent, that is effective to treat the cancer. In one aspect, the cancer is a HER2-expressing cancer (e.g., breast or ovarian cancer) and the CPMV or CP conjugate further comprises the HER2-ligand or a fragment thereof. The anticancer agent is selected to be effective against the cancer being treated. The subject can be a mammal, e.g., a canine, feline, equine, bovine or a human. The therapy can be combined with other anti-cancer therapies, e.g., tumor resection or immunotherapy and can be a first line, second line, third line, fourth line or fifth line therapy. When used as a combination therapy, the conjugates can comprise the same or different agents and thus, in one aspect can be used to administer a combination therapy to the subject. One of skill in the art can determine the effective amount to be delivered and will know when the methods has been effective by detecting clinical or subject clinical indications of treatment, e.g., reduction in tumor burden, reduction in cancer markers, or longer progression free survival or remission. Alternatives to CPMV or CP include tobacco mosaic virus, tobacco mild green mottle virus, potato virus, and physalis mottle virus.

Also provided are a plurality of the conjugates as provided herein. The conjugates are the same or different from each other. The plurality can be used as described herein and can be used to delivery different detectable labels or agents.

Also provided is a composition comprising the conjugate or the plurality thereof and a carrier such as a pharmaceutically acceptable carrier.

Also provided herein is a method for treating a HER-2 expressing cancer in a subject in need thereof comprising, or consisting essentially of, or yet further consisting of administering an effective amount of a conjugate wherein the lysine of the peptide is chemically modified to comprise a human epidermal growth factor receptor 2 (HER2)-specific targeting peptide ligand (e.g., the peptide FCDGFYACYMDV) (SEQ. ID NO: 6) to the subject. In a further aspect, the HER-2 expressing cancer may be resistant to chemotherapy. In one aspect the cancer is a breast cancer cell or an ovarian cancer cell. In a further aspect, the ovarian cancer cell or breast cancer cell may be resistant to chemotherapy. In a further aspect, the subject is a mammal, e.g., a murine, a rat, a canine, a feline or a human. The therapy can be combined with other cancer therapies, e.g., tumor ablation or chemotherapy and can be a first line, second line, third line, fourth line or fifth line therapy.

### BRIEF DESCRIPTION OF THE DRAWINGS

FIGS. 1A-1E: Characterization of CPMV. (FIG. 1A) Denaturing sodium dodecyl sulfate polyacrylamide gel electrophoresis (SDS-PAGE). Lane 1=SeeBlue™ Plus2 Pre-stained Protein Standard; Lane 2=CPMV. Black triangle=large coat protein (42 kDa); Open triangle=small coat protein (24 kDa). (FIG. 1B) Native agarose gel electrophoresis of CPMV where the same gel was stained with RNA stain GelRed (nucleic acid) and protein stain Coomassie Brilliant Blue (protein stain). (FIG. 1C) Negatively stained CPMV was analyzed by transmission electron microscope (TEM). (FIG. 1D) Triplicate samples of CPMV were analyzed by dynamic light scattering (DLS), and the representative data sets are shown. D refers to average diameter of particles; PDI refers to polydispersity index. (FIG. 1E) CPMV analyzed by size exclusion chromatography (SEC) using a Superose 6 column. Nucleic acids were detected at 260 nm, and protein was detected at 280 nm.

FIGS. 2A-2E: Isolation and validation of CPMV binding monoclonal phages. (FIG. 2A) Monoclonal ELISA of monoclonal phages against CPMV (red dots) and BSA (green dots). CPMV only (blue dot) served as negative control to validate the binding of HRP-conjugated anti-M13 monoclonal antibody against CPMV. (FIG. 2B) Sequences of CPMV binding peptides (CBPs) identified by DNA sequencing. (FIG. 2C) Heatmap showing cross-reactivity of monoclonal phages against CPMV vs. other viruses. TMV: tobacco mosaic virus. TMGMV: tobacco mild green mosaic virus. PVX: potato virus X. PhMV: Physalis mottle virus. CCMV: cowpea chlorotic mottle virus. BSA: bovine serum albumin (negative control). (FIG. 2D) Titration ELISA comparing binding of monoclonal phages across a serial concentration of CPMV.MP_{SEF}: GWRVSEF; MP_{SEL}: GWRVSEL; MP_{SLH}: GFHYSLH (FIG. 2E) Dot blot to determine the specific binding of monoclonal phages (MP_{SEF}) to CPMV. (1) MP_{SEF} coated, probed with CPMV. (2) M13KE (empty phage) coated, probed with CPMV. (3) CPMV only (positive control). (4) BSA coated, probed with CPMV. (5) only. (6) M13KE only. All samples were then incubated with primary antibodies (goat anti-CPMV rabbit polyclonal antibody), and subsequently with secondary antibodies (HRP conjugated goat anti-rabbit antibodies).

FIGS. 3A-3D: Binding of CPMV-binding peptide (CBP) to CPMV. (FIG. 3A) Characterization of CPMV with non-covalent display peptides CBP-FITC and CBP-biotin. (FIG. 3B) Native agarose gel electrophoresis. (Lane 1) CPMV only. (Lane 2) CPMV with CBP-FITC. (Lane 3) CBP-FITC only. (FIG. 3C) Western blot analysis. CPMV on membrane was detected with (Lane 1) rabbit anti-CPMV polyclonal antibodies, followed by goat anti-rabbit monoclonal antibody conjugated with horseradish peroxidase (HRP); (Lane 2) CBP-biotin, followed by streptavidin HRP; (Lane 3) streptavidin HRP. Black arrow indicates CPMV large coat protein (˜42 kDa), and open arrow indicates CPMV small coat protein (˜24 kDa). M represents SeeBlue™ Plus2 Pre-stained Protein Standard. (FIG. 3D) Representative images from TEM with immunogold labeling. CPMV-CBP-biotin complex was stained with streptavidin-labeled with gold nanoparticles (5 nm); indicated by red arrows. Non-specific binding of streptavidin-labeled gold nanoparticles to CPMV only was not observed.

FIGS. 4A-4H Enzyme-linked immunosorbent assay (ELISA) of CBP binding to CPMV. Cartoon (FIG. 4A-4D): (FIG. 4E) ELISA of CBP-biotin. (Sample) CBP-biotin coated onto streptavidin well followed by addition of CPMV; CPMV was omitted for the control group. This was followed by detection using rabbit anti-CPMV polyclonal antibodies followed by HRP-conjugated goat anti-rabbit monoclonal antibodies. Results were compared using unpaired t-test (with ****=p<0.0001). (FIG. 4F) Titration ELISA of CBP-FITC against a range of CPMV concentrations. (FIG. 4G) Competitive ELISA between CBP-biotin and MP_{SEF}. (FIG. 4H) Competitive ELISA between CBP-FITC and MP_{SEF}. The half of maximal inhibitory concentration (IC50) value was determined by GraphPad Prism using nonlinear regression analysis (One site-Fit log IC50).

FIG. 5: Validation of CBP-AHNP binding to CPMV. Top panel: cartoon of peptides used; illustration of CPMV with CBP-AHNP and *AHNP. *AHNP without CBP domain acts as control and should not bind to CPMV; and bottom panel: Native agarose gel electrophoresis. (Lane 1) CPMV only. (Lane 2) CPMV-CBP-AHNP. (3) CPMV-*AHNP. (4) CBP-AHNP only. (5) *AHNP only.

FIGS. 6A-6D: CPMV-CBP-AHNP cell binding assays. Flow cytometry analysis was performed to assess cell uptake of CPMV, CPMV-*AHNP, and CPMV-CBP-AHNP particles by murine macrophages, RAW264.7 cells (FIG. 6A); as well as cell binding of the nanoparticle formulations to ovarian cancer cells, SKOV3 (FIG. 6B). Histograms and bar charts showing median fluorescent intensity (MFI) and % positive cells for the APC channel (Cy5 signal from CPMV staining) and FITC channel (FITC signal from labeled peptides) of RAW264.7 cells (FIG. 6C); and SKOV3 (FIG. 6D). Error bars indicate standard deviation. Statistical analysis was determined by one-way ANOVA with Tukey's test: ns p>0.05, *p≤0.05, **p≤0.01, ***p≤0.001, ***p≤0.0001

FIG. 7: Characterization of virus particles with TEM imaging and 12% SDS-PAGE. The molecular weight of coat protein of each viruses: TMV: tobacco mosaic virus (17.5 kDa); TMGMV: tobacco mild green mottle virus (17.5 kDa); PVX: potato virus X (25 kDa); PhMV: physalis mottle virus virus-like particle (21 kDa); CCMV: cowpea mottle virus (20 kDa). The virus preparation of TMV, TMGMV, and CCMV were pure by showing only one respective band on SDS-PAGE gel except for PVX and PhMV virus-like particle. The occurrence of double bands for PVX is due to proteolytic cleavage during purification. However, this cleavage does not affect the particle's activity nor structural integrity.⁴⁶ Slight protein contaminants were observed in PhMV preparation that might be contributed by E. coli proteins from purification.

FIG. 8: Native agarose gel electrophoresis of CBP-FITC. Lane 1 and Lane 2 shows the end product after CBP-FITC was purified with dual purification using desalting columns contains G-25 resin followed by 100 kDa molecular weight cut-off centrifugal filter membrane. Lane 3 shows CBP-FITC only without any purification steps.

FIG. 9—Native agarose gel electrophoresis of CBP-FITC comparing the effectiveness of single-step purification (either desalting or filtration) vs. dual-step purification of CBP-FITC. Trace amount of CBP-FITC was spotted after single purification by sephadex desalting columns (G-25 resin). Here 75 μg of CBP-FITC was considered; this amount of CBP-FITC was adjusted to match the amounts used during labeling of CPMV (using a 500-fold molar excess of CBP-FITC to 0.5 mg of CPMV at 1 mg/ml).

### DETAILED DESCRIPTION

Unless defined otherwise, all technical and scientific terms used herein have the same meanings as commonly understood by one of ordinary skill in the art to which this disclosure belongs. All nucleotide sequences provided herein are presented in the 5′ to 3′ direction. All polypeptide and protein sequences are presented in the direction of the amine terminus to carboxy terminus. Although any methods and materials similar or equivalent to those described herein can be used in the practice or testing of the present disclosure, particular, non-limiting exemplary methods, devices, and materials are now described. All technical and patent publications cited herein are incorporated herein by reference in their entirety. Nothing herein is to be construed as an admission that the disclosure is not entitled to antedate such disclosure by virtue of prior invention.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds, (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

All numerical designations, e.g., pH, temperature, time, concentration, and molecular weight, including ranges, are approximations which are varied (+) or (−) by increments of 1.0 or 0.1, as appropriate or alternatively by a variation of +/−15%, or alternatively 10% or alternatively 5% or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

As used in the specification and claims, the singular form “a”, “an” and “the” include plural references unless the context clearly dictates otherwise. For example, the term “a polypeptide” includes a plurality of polypeptides, including mixtures thereof.

As used herein, “and/or” refers to and encompasses any and all possible combinations of one or more of the associated listed items, as well as the lack of combinations when interpreted in the alternative (“or”).

“Optional” or “optionally” means that the subsequently described circumstance may or may not occur, so that the description includes instances where the circumstance occurs and instances where it does not.

As used herein, the term “comprising” is intended to mean that the compositions and methods include the recited elements, but do not exclude others. “Consisting essentially of” when used to define compositions and methods, shall mean excluding other elements of any essential significance to the combination for the intended use. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions disclosed herein. Embodiments defined by each of these transition terms are within the scope of this disclosure.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, the term “animal” refers to living multi-cellular vertebrate organisms, a category that includes, for example, mammals and birds. The term “mammal” includes both human and non-human mammals.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has or is diagnosed of having or is suspected of having a disease.

As used herein, the terms “treating,” “treatment” and the like are used herein to mean obtaining a desired pharmacologic and/or physiologic effect. In some embodiments, the effect can be prophylactic in terms of completely or partially preventing a disorder or sign or symptom thereof, and/or can be therapeutic in terms of a partial or complete cure for a disorder and/or adverse effect attributable to the disorder. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, e.g., a cancer such as breast cancer. In some embodiments, they refer to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. When the disease is cancer, the following clinical end points are non-limiting examples of treatment: reduction in tumor burden, slowing of tumor growth, longer overall survival, and longer time to tumor progression, inhibition of metastasis or a reduction in metastasis of the tumor. In one aspect, treatment excludes prophylaxis.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

In one embodiment, the term “disease” or “disorder” as used herein refers to a cancer or a tumor (which are used interchangeably herein), a status of being diagnosed with such disease, a status of being suspect of having such disease, or a status of at high risk of having such disease.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features.

A “solid tumor” is an abnormal mass of tissue that usually does not contain cysts or liquid areas. Solid tumors can be benign or malignant. Different types of solid tumors are named for the type of cells that form them. Examples of solid tumors include, but not limited to, sarcomas, carcinomas, and lymphomas. In some embodiments, a solid tumor comprises bladder cancer, bone cancer, brain cancer, breast cancer, colorectal cancer, esophageal cancer, eye cancer, head and neck cancer, kidney cancer, lung cancer, melanoma, ovarian cancer, pancreatic cancer, prostate cancer, skin cancer, gastric cancer, esophageal cancer, colon cancer, glioma, cervical cancer, hepatocellular, thyroid cancer, or stomach cancer.

As used herein, a “metastatic cancer” is a cancer that spreads from where it originated to another part of the body.

As used herein, a “cancer cell” are cells that have uncontrolled cell division and form solid tumors or enter the blood stream.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited on May 1, 2008. Typically, a patient is given a subsequent chemotherapy regimen because the patient did not show a positive clinical or sub-clinical response to the first line therapy or the first line therapy has stopped.

The term “conjugation” or “bioconjugation” as used herein refers to a chemical technique used to couple two molecules together, at least one of which is a biomolecule, such as a carbohydrate, nucleic acid, or protein. Exemplary reactions include, but are not limited to, cysteine-disulfide linkages, cysteine-maleimide linkages, or lysine-isocyanate linkages.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment disclosed herein that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “protein”, “peptide” and “polypeptide” are used interchangeably and in their broadest sense to refer to a compound of two or more subunit amino acids, amino acid analogs or peptidomimetics. The subunits (which are also referred to as residues) may be linked by peptide bonds. In another embodiment, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “plasmid” is an extra-chromosomal DNA molecule separate from the chromosomal DNA which is capable of replicating independently of the chromosomal DNA. In many cases, it is circular and double-stranded. Plasmids provide a mechanism for horizontal gene transfer within a population of microbes and typically provide a selective advantage under a given environmental state. Plasmids may carry genes that provide resistance to naturally occurring antibiotics in a competitive environmental niche, or alternatively the proteins produced may act as toxins under similar circumstances.

“Plasmids” used in genetic engineering are called “plasmid vectors”. Many plasmids are commercially available for such uses. The gene to be replicated is inserted into copies of a plasmid containing genes that make cells resistant to particular antibiotics and a multiple cloning site (MCS, or polylinker), which is a short region containing several commonly used restriction sites allowing the easy insertion of DNA fragments at this location. Another major use of plasmids is to make large amounts of proteins. In this case, researchers grow bacteria containing a plasmid harboring the gene of interest. Just as the bacterium produces proteins to confer its antibiotic resistance, it can also be induced to produce large amounts of proteins from the inserted gene. This is a cheap and easy way of mass-producing a gene or the protein it then codes for.

“HER2” refers to receptor tyrosine-protein kinase erbB-2, also known as CD340. Amplification or over-expression of this oncogene has been shown to play an important role in the development and progression of certain aggressive types of breast cancer. In recent years the protein has become an important biomarker and target of therapy for approximately 30% of breast cancer patients.

Cowpea mosaic virus (CPMV) is a plant-infecting member of the order Picornavirales, with a relatively simple, non-enveloped capsid that has been extensively studied and a positive-sense, single-stranded RNA genome. For CPMV, the genome is bipartite, with RNA-1 (6 kb) and RNA-2 (3.5 kb) being separately encapsidated. CPMV has an icosahedral capsid structure, which is ˜30 nm in diameter and is formed from 60 copies each of a Large (L) and Small (S) coat protein. These two coat proteins are processed from a single RNA-2-encoded precursor polyprotein (VP60) by the action of the 24 K viral proteinase which is encoded by RNA-1. Thus capsid assembly, as well as viral infection, is dependent on the presence of both genomic segments in an infected plant cell.

The terms “CPMV” “CPMV virus” or “CPMV particles” are used interchangeably, referring to a CPMV comprising, or alternatively consisting essentially of, or yet consisting of a capsid and an RNA genome (which is also referred to herein as a viral genome) encapsidated in the capsid. In some embodiments, the CPMV particles have been treated, prepared and/or inactivated by a method as disclosed herein. In some embodiments, the CPMV particle further comprises a heterologous RNA, which is heterologous to (i.e., not naturally presented in) a native CPMV free of any human intervention. Although not explicitly stated, tobacco mosaic virus, tobacco mild green mottle virus, potato virus, and physalis mottle virus virus-like particle provide alternatives to the use of CPMV.

The virus can be obtained according to various methods known to those skilled in the art. In embodiments where plant virus particles are used, the virus particles can be obtained from the extract of a plant infected by the plant virus. For example, cowpea mosaic virus can be grown in black eyed pea plants, which can be infected within 10 days of sowing seeds. Plants can be infected by, for example, coating the leaves with a liquid containing the virus, and then rubbing the leaves, preferably in the presence of an abrasive powder which wounds the leaf surface to allow penetration of the leaf and infection of the plant. Within a week or two after infection, leaves are harvested and viral nanoparticles are extracted. In the case of cowpea mosaic virus, 100 mg of virus can be obtained from as few as 50 plants. Procedures for obtaining plant picornavirus particles using extraction of an infected plant are known to those skilled in the art. See Wellink J., Meth Mol Biol, 8, 205-209 (1998). Procedures are also available for obtaining virus-like particles. Saunders et al., Virology, 393(2):329-37 (2009). The disclosures of both of these references are incorporated herein by reference.

As used herein, an anticancer agent refers to any drug or compound used for anticancer treatment. These include any drug that renders or maintains a clinical symptom or diagnostic marker of tumors and cancer, alone or in combination with other compounds, that reduces or maintains a state of remission, reduction, remission, prevention or remission. In some embodiments, the agent is an RNA and/or a DNA. In some embodiments, the agent is a protein or a polypeptide. In some embodiments, the agent is a chemical compound. Examples of anticancer agents include angiogenesis inhibitors such as angiostatin Kl-3, DL-adifluoromethyl-ornithine, endostatin, fumagillin, genistein, minocycline, staurosporine, and (+)-thalidomide; DNA intercalating or cross-linking agents such as bleomycin, carboplatin, carmustine, chlorambucil, cyclophosphamide, cisplatin, melphalan, mitoxantrone, and oxaliplatin; DNA synthesis inhibitors such as methotrexate, 3-Amino-1,2,4-benzotriazine 1,4-dioxide, aminopterin, cytosine b-D-arabinofuranoside, 5-Fluoro-5′-deoxyuridine, 5-Fluorouracil, gaciclovir, hydroxyurea, and mitomycin C; DNA-RNA transcription regulators such as actinomycin D, daunorubicin, doxorubicin, homoharringtonine, and idarubicin; enzyme inhibitors such as S(+)-camptothecin, curcumin, (−)-deguelin, 5,6-dichlorobenzimidazole I-b-D-ribofuranoside, etoposine, formestane, fostriecin, hispidin, cyclocreatine, mevinolin, trichostatin A, tyrophostin AG 34, and tyrophostin AG 879, Gene Regulating agents such as 5-aza-2′-deoxycitidine, 5-azacytidine, cholecalciferol, 4-hydroxytamoxifen, melatonin, mifepristone, raloxifene, all trans-retinal, all trans retinoic acid, 9-cis-retinoic acid, retinol, tamoxifen, and troglitazone; Microtubule Inhibitors such as colchicine, dolostatin 15, nocodazole, paclitaxel, podophyllotoxin, rhizoxin, vinblastine, vincristine, vindesine, and vinorelbine; humanised or mouse/human chimeric monoclonal antibodies against defined cancer associated structures (such as Trastuzumab, Rituximab, Cetuximab, Bevacizumab, Alemtuzumab); and various other antitumor agents such as 17-(allylamino)-17-demethoxygeldanamycin, 4-Amino-1,8-naphthalimide, apigenin, brefeldin A, cimetidine, dichloromethylene-diphosphonic acid, leuprolide, luteinizing-hormone-releasing hormone, pifithrin-a, rapamycin, thapsigargin, and bikunin, and derivatives (as defined for imaging agents) thereof.

As used herein, an ablative therapy is a treatment destroying or ablating cancer tumors. In one embodiment, the ablative therapy does not require invasive surgery. In other embodiments, the ablative therapy refers to removal of a tumor via surgery. In some embodiments, the step ablating the cancer includes immunotherapy of the cancer. Cancer immunotherapy is based on therapeutic interventions that aim to utilize the immune system to combat malignant diseases. It can be divided into unspecific approaches and specific approaches. Unspecific cancer immunotherapy aims at activating parts of the immune system generally, such as treatment with specific cytokines known to be effective in cancer immunotherapy (e.g. IL-2, interferon's, cytokine inducers).

In some embodiments, a method as disclosed herein further includes the step of ablating the cancer. Ablating the cancer can be accomplished using a method selected from the group consisting of cryoablation, thermal ablation, radiotherapy, chemotherapy, radiofrequency ablation, electroporation, alcohol ablation, high intensity focused ultrasound, photodynamic therapy, administration of monoclonal antibodies, immunotherapy, and administration of immunotoxins.

A “composition” is intended to mean a combination of active agent and another compound or composition, inert (for example, a detectable agent or label) or active, such as an adjuvant, diluent, binder, stabilizer, buffers, salts, lipophilic solvents, preservative, adjuvant or the like and include pharmaceutically acceptable carriers.

Carriers also include pharmaceutical excipients and additives proteins, peptides, amino acids, lipids, and carbohydrates (e.g., sugars, including monosaccharides, di-, tri, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, arginine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

A “pharmaceutical composition” is intended to include the combination of an active agent with a carrier, inert or active, making the composition suitable for diagnostic or therapeutic use in vitro, in vivo or ex vivo.

“Pharmaceutically acceptable carriers” refers to any diluents, excipients, or carriers that may be used in the compositions disclosed herein. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They may be selected with respect to the intended form of administration, that is, oral tablets, capsules, elixirs, syrups and the like, and consistent with conventional pharmaceutical practices.

The compositions used in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions are administered in a manner compatible with the dosage formulation and in such amount as is therapeutically or prophylactically effective. The formulations are easily administered in a variety of dosage forms, such as the type of injectable solutions described herein.

As used herein, the term “contacting” means direct or indirect binding or interaction between two or more molecules. A particular example of direct interaction is binding. A particular example of an indirect interaction is where one entity acts upon an intermediary molecule, which in turn acts upon the second referenced entity. Contacting as used herein includes in solution, in solid phase, in vitro, ex vivo, in a cell and in vivo. Contacting in vivo can be referred to as administering, or administration.

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the optimal route will vary with the condition and age of the recipient, and the disease being treated.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents disclosed herein for any particular subject depends upon a variety of factors including the activity of the specific compound employed, bioavailability of the compound, the route of administration, the age of the animal and its body weight, general health, sex, the diet of the animal, the time of administration, the rate of excretion, the drug combination, and the severity of the particular disorder being treated and form of administration. In general, one will desire to administer an amount of the compound that is effective to achieve a serum level commensurate with the concentrations found to be effective in vivo. These considerations, as well as effective formulations and administration procedures are well known in the art and are described in standard textbooks.

“Therapeutically effective amount” of a drug or an agent refers to an amount of the drug or the agent that is an amount sufficient to obtain a pharmacological response such as passive immunity; or alternatively, is an amount of the drug or agent that, when administered to a patient with a specified disorder or disease, is sufficient to have the intended effect, e.g., treatment, alleviation, amelioration, palliation or elimination of one or more manifestations of the specified disorder or disease in the patient. A therapeutic effect does not necessarily occur by administration of one dose, and may occur only after administration of a series of doses. Thus, a therapeutically effective amount may be administered in one or more administrations.

As used herein, the phrase “immune response” or its equivalent “immunological response” refers to the development of a cell-mediated response (e.g. mediated by antigen-specific T cells or their secretion products). A cellular immune response is elicited by the presentation of polypeptide epitopes in association with Class I or Class II MHC molecules, to treat or prevent a viral infection, expand antigen-specific B-reg cells, TC1, CD4+ T helper cells and/or CD8+ cytotoxic T cells and/or disease generated, autoregulatory T cell and B cell “memory” cells. The response may also involve activation of other components. In some aspect, the term “immune response” may be used to encompass the formation of a regulatory network of immune cells. Thus, the term “regulatory network formation” may refer to an immune response elicited such that an immune cell, preferably a T cell, more preferably a T regulatory cell, triggers further differentiation of other immune cells, such as but not limited to, B cells or antigen-presenting cells—non-limiting examples of which include dendritic cells, monocytes, and macrophages. In certain embodiments, regulatory network formation involves B cells being differentiated into regulatory B cells; in certain embodiments, regulatory network formation involves the formation of tolerogenic antigen-presenting cells.

The term “immune cells” includes, e.g., white blood cells (leukocytes) which are derived from hematopoietic stem cells (HSC) produced in the bone marrow, lymphocytes (T cells, B cells, natural killer (NK) cells) and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), natural killer T-cells, T-regulatory cells (Treg) and gamma-delta T cells. A “cytotoxic cell” includes CD8+ T cells, natural-killer (NK) cells, and neutrophils, which cells are capable of mediating cytotoxicity responses. Cytokines are small secreted proteins released by immune cells that have a specific effect on the interactions and communications between the immune cells. Cytokines can be pro-inflammatory or anti-inflammatory. Non-limiting example of a cytokine is Granulocyte-macrophage colony-stimulating factor (GM-CSF), which stimulates stem cells to produce granulocytes (neutrophils, eosinophils, and basophils) and monocytes.

### Modes for Carrying Out the Disclosure

### Conjugates

Applicant provides herein a conjugate and compositions containing same comprising a peptide (e.g., GWRVSEF) (SEQ ID NO: 1)) with an optional C-terminal lysine, that binds to CPMV or CP, that is useful in one aspect to display desired molecules on CPMV or CP. Alternatives to CPMV for use in this disclosure include tobacco mosaic virus, tobacco mild green mottle virus, potato virus, and physalis mottle virus.

The peptide can be loaded with molecules not limited to biotin, fluorophore, protein/peptide or other moieties that can be modified with the CPMV-binding peptide, such as for example, small drugs, imaging agents, nucleic acids etc. Alternatives to CPMV include tobacco mosaic virus, tobacco mild green mottle virus, potato virus, and physalis mottle virus. Depending on the molecule conjugated to binding peptide (GWRVSEF) (SEQ. ID NO: 1), useful activities ranging from cancer targeting, cardiovascular targeting—any tissue targeting, and payload delivery of therapeutics (small molecule, protein), contrast agents for imaging, etc. Thus, also provided is a method to display the desired molecules on a CPMV by contacting the CPMV with the peptide as disclosed.

Also provided herein is a conjugate or composition containing same comprising at least one peptide selected from: N-GWRVSEF-C(SEQ ID NO: 1), N-GWRVSEL-C(SEQ ID NO: 2), N-GWRVSE-C(SEQ ID NO: 3), N-GFHYSLH-C(SEQ ID NO: 4), or N-IVGSQVT-C (SEQ ID NO: 5) conjugated to the surface of a cowpea mosaic virus (CPMV) or CMPV coat protein (CP) wherein the N-terminus of the peptide is linked to the CPMV surface. Alternatives to CPMV or CP include tobacco mosaic virus, tobacco mild green mottle virus, potato virus, and physalis mottle virus. In one aspect, the conjugate further comprises a lysine at the C-terminus of the peptide. In another aspect, the lysine is chemically modified to further comprise one or more of a peptide, FITC, a ligand, biotin, fluorophore, an anti-cancer agent, or a human epidermal growth factor receptor 2 (HER2)-specific targeting peptide ligand. In another aspect, the conjugate further comprises an active agent conjugated to the exterior or interior surface of the CPMV or CP by covalent coupling of the active agents to the coat proteins of the CPMV.

In a further aspect, the CPMV or CP and therefore the conjugate as provided herein is chemically modified by a method comprising one or more of bioconjugation, targeting solvent-exposed lysine side chains using N-hydroxysuccinimidyl ester (NHS) reaction and biorthogonal click-chemistry such as copper-catalyzed azide-alkyne cycloaddition (CuAAC). Alternatives to CPMV include tobacco mosaic virus, tobacco mild green mottle virus, potato virus, and physalis mottle virus.

In another aspect, the interior of the CPMV or CP is modified by one or more of targeting cysteine side chains on the interior capsid surface or targeting surface loops. Alternatives to CPMV or CP include tobacco mosaic virus, tobacco mild green mottle virus, potato virus, and physalis mottle virus.

Thus, also provided is a method to link the conjugate to a target (e.g., cell, ligand or receptor) CPMV or CP conjugate with the target as disclosed by contacting the target with the CPMV conjugate. These methods are useful for cell labeling, diagnostics and therapies. One of skill in the art can determine when the target has been linked to the conjugate by methods known to the skilled artisan and briefly described herein, e.g., by detection of the fluorophore or when the conjugate delivers an agent, if the cell or target has been modified by the conjugate, e.g., the growth of a cancer cell has been inhibited by the delivery of an anti-cancer drug or therapy. The contacting of the cell in vitro or in vivo. The cell can be from a mammal, e.g., a human cell. When contacted in vivo, the method is useful to test the effectiveness of the therapy against a cancer cell. The cancer can be obtained from a biopsy or an established cell line. These are commercially available from vendors, such as the American Type Culture Collection (ATCC). When practiced in vivo, the method is useful as an animal model to test combination therapies or to test the therapy against cancer types.

In another aspect, the conjugate further comprises an active agent conjugated to the exterior or interior surface of the CPMV or CP by covalent coupling of the active agents to the coat proteins of the CPMV. Alternatives to CPMV or CP include tobacco mosaic virus, tobacco mild green mottle virus, potato virus, and physalis mottle virus. Thus, also provided is a method to deliver to a target (e.g., cell, ligand or receptor) active agent to the target as disclosed by contacting the CPMV or CP conjugate with the cell. These methods are useful for cell labeling, diagnostics and therapies. One of skill in the art can determine when the target has been delivered by methods know to the skilled artisan and briefly described herein, e.g., by determining if the cell or target has been modified by the conjugate, e.g., the growth of a cancer cell has been inhibited by the delivery of an anti-cancer drug or therapy. The contacting of the cell in vitro or in vivo. The cell can be from a mammal, e.g., a human cell. When contacted in vivo, the method is useful to test the effectiveness of the therapy for effectiveness against a cancer cell or for cell labeling. The cancer can be obtained from a biopsy or an established cell line. These are commercially available from vendors, such as the American Type Culture Collection (ATCC).

In another aspect, the conjugate further comprises an agent infused into the CPMV or CP of the conjugate. In one aspect, the CPMV or CP is modified as described herein. Thus, also provided is a method to deliver to a target (e.g., cell, ligand or receptor) active agent to the target as disclosed by contacting the CPMV or CP conjugate with the agent with the cell or tissue. Alternatives to CPMV or CP include tobacco mosaic virus, tobacco mild green mottle virus, potato virus, and physalis mottle virus. These methods are useful for cell labeling, diagnostics and therapies. One of skill in the art can determine when the target has been delivered by methods know to the skilled artisan and briefly described herein, e.g., by determining if the cell or target has been modified by the conjugate, e.g., the growth of a cancer cell has been inhibited by the delivery of an anti-cancer drug or therapy. The contacting of the cell in vitro or in vivo. The cell can be from a mammal, e.g., a human cell. The cells can be obtained from a biopsy or an established cell line. These are commercially available from vendors, such as the American Type Culture Collection (ATCC).

In a further aspect, the lysine of the peptide is chemically modified to further comprise a human epidermal growth factor receptor 2 (HER2)-specific targeting peptide ligand. In another aspect, the ligand comprises the peptide FCDGFYACYMDV (SEQ ID NO: 6). In one aspect, the CPMV or CP of the conjugate is modified to comprise or deliver an active or detectable agent, e.g., an agent useful to inhibit the growth or detect the HER2-expressing cell such as a cancer cell. Alternatives to CPMV or CP include tobacco mosaic virus, tobacco mild green mottle virus, potato virus, and physalis mottle virus. When the conjugate is used to deliver an anti-cancer agent, the CPMV or comprises the agent specific for the cell being treated, e.g. an anti-breast cancer drug to treat breast cancer. These conjugates are useful to inhibit the growth of a HER2 expressing cancer cell (e.g., breast cancer cell or ovarian cancer cells) by contacting the cell in vitro or in vivo. The cell can be from a mammal, e.g., a human cell. When contacted in vivo, the method is useful to test the effectiveness of the therapy for effectiveness against a cancer cell. The cancer can be obtained from a biopsy or an established cell line. These are commercially available from vendors, such as the American Type Culture Collection (ATCC). When practiced in vivo, the method is useful as an animal model to test combination therapies or to test the therapy against cancer types.

### Compositions

In another aspect, provided herein is a composition comprising, consisting essentially of, or consisting of the combination of formulations comprising a conjugate as provided herein, and at least one carrier, such as a pharmaceutically acceptable carrier or excipient. In one aspect, the composition further comprises a preservative or stabilizer.

In one embodiment, this technology relates to a composition comprising a combination of conjugates or formulations as described herein and a carrier.

In another embodiment, this disclosure provides a pharmaceutical composition comprising an effective amount or a therapeutically effective amount of a conjugate formulations as described herein and a pharmaceutically acceptable carrier.

Compositions, including pharmaceutical compositions comprising, consisting essentially of, or consisting of the conjugate alone or in combination of other therapeutic agents can be manufactured by means of conventional mixing, dissolving, granulating, dragee-making levigating, emulsifying, encapsulating, entrapping, or lyophilization processes. These can be formulated in conventional manner using one or more physiologically acceptable carriers, diluents, excipients, or auxiliaries which facilitate processing of the combinations of compounds provided herein into preparations which can be used pharmaceutically.

In some embodiments, the pharmaceutical formulations described herein are administered to a subject by multiple administration routes, including but not limited to, parenteral, oral, buccal, rectal, sublingual, or transdermal administration routes. In some cases, parenteral administration comprise, or consists essentially of, or yet further consists of, intravenous, subcutaneous, intramuscular, intracerebral, intranasal, intra-arterial, intra-articular, intradermal, intravitreal, intraosseous infusion, intraperitoneal, or intrathecal administration. In some instances, the pharmaceutical composition is formulated for local administration. In other instances, the pharmaceutical composition is formulated for systemic administration.

In some embodiments, the pharmaceutical formulations include, but are not limited to, lyophilized formulations, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations (e.g., nanoparticle formulations), and mixed immediate and controlled release formulations.

In some embodiments, the pharmaceutical formulations include a carrier or carrier materials selected on the basis of compatibility with the composition disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like. Pharmaceutically compatible carrier materials include, but are not limited to, acacia, gelatin, colloidal silicon dioxide, calcium glycerophosphate, calcium lactate, maltodextrin, glycerine, magnesium silicate, polyvinylpyrrollidone (PVP), cholesterol, cholesterol esters, sodium caseinate, soy lecithin, taurocholic acid, phosphotidylcholine, sodium chloride, tricalcium phosphate, dipotassium phosphate, cellulose and cellulose conjugates, sugars sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995), Hoover, John E., Remington's Pharmaceutical Sciences, Mack Publishing Co., Easton, Pennsylvania 1975, Liberman, H. A. and Lachman, L., Eds., Pharmaceutical Dosage Forms, Marcel Decker, New York, N.Y., 1980, and Pharmaceutical Dosage Forms and Drug Delivery Systems, Seventh Ed. (Lippincott Williams & Wilkins 1999).

In some instances, the pharmaceutical formulations further include pH adjusting agents or buffering agents which include acids such as acetic, boric, citric, lactic, phosphoric and hydrochloric acids, bases such as sodium hydroxide, sodium phosphate, sodium borate, sodium citrate, sodium acetate, sodium lactate and tris-hydroxymethylaminomethane, and buffers such as citrate/dextrose, sodium bicarbonate and ammonium chloride. Such acids, bases and buffers are included in an amount required to maintain pH of the composition in an acceptable range.

In some instances, the pharmaceutical formulation includes one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions, suitable salts include sodium chloride, potassium chloride, sodium thiosulfate, sodium bisulfite and ammonium sulfate.

In some embodiments, the pharmaceutical formulations include, but are not limited to, sugars like trehalose, sucrose, mannitol, maltose, glucose, or salts like potassium phosphate, sodium citrate, ammonium sulfate and/or other agents such as heparin to increase the solubility and in vivo stability of polypeptides.

In some instances, the pharmaceutical formulations further include diluent which are used to stabilize compounds because they can provide a more stable environment. Salts dissolved in buffered solutions (which also can provide pH control or maintenance) are utilized as diluents in the art, including, but not limited to a phosphate buffered saline solution. In certain instances, diluents increase bulk of the composition to facilitate compression or create sufficient bulk for homogenous blend for capsule filling. Such compounds can include e.g., lactose, starch, mannitol, sorbitol, dextrose, microcrystalline cellulose such as AVICEL®, dibasic calcium phosphate, dicalcium phosphate dihydrate, tricalcium phosphate, calcium phosphate, anhydrous lactose, spray-dried lactose, pregelatinized starch, compressible sugar, such as Di-PAC® (Amstar), mannitol, hydroxypropylmethylcellulose, hydroxypropylmethylcellulose acetate stearate, sucrose-based diluents, confectioner's sugar, monobasic calcium sulfate monohydrate, calcium sulfate dihydrate, calcium lactate trihydrate, dextrates, hydrolyzed cereal solids, amylose, powdered cellulose, calcium carbonate, glycine, kaolin, mannitol, sodium chloride, inositol, bentonite, and the like.

In some cases, the pharmaceutical formulations include disintegration agents or disintegrants to facilitate the breakup or disintegration of a substance. The term “disintegrate” include both the dissolution and dispersion of the dosage form when contacted with gastrointestinal fluid. Examples of disintegration agents include a starch, e.g., a natural starch such as corn starch or potato starch, a pregelatinized starch such as National 1551 or AMIJEL®, or sodium starch glycolate such as PROMOGEL® or EXPLOTAB®, a cellulose such as a wood product, methylcrystalline cellulose, e.g., AVICEL®, AVICEL® PH101, AVICEL® PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose, a cross-linked starch such as sodium starch glycolate, a cross-linked polymer such as crospovidone, a cross-linked polyvinylpyrrolidone, alginate such as alginic acid or a salt of alginic acid such as sodium alginate, a clay such as VEEGUM® HV (magnesium aluminum silicate), a gum such as agar, guar, locust bean, Karaya, pectin, or tragacanth, sodium starch glycolate, bentonite, a natural sponge, a surfactant, a resin such as a cation-exchange resin, citrus pulp, sodium lauryl sulfate, sodium lauryl sulfate in combination starch, and the like.

In some instances, the pharmaceutical formulations include filling agents such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

Lubricants and glidants are also optionally included in the pharmaceutical formulations described herein for preventing, reducing or inhibiting adhesion or friction of materials.

Exemplary lubricants include, e.g., stearic acid, calcium hydroxide, talc, sodium stearyl fumerate, a hydrocarbon such as mineral oil, or hydrogenated vegetable oil such as hydrogenated soybean oil (STEROTEX®), higher fatty acids and their alkali-metal and alkaline earth metal salts, such as aluminum, calcium, magnesium, zinc, stearic acid, sodium stearates, glycerol, talc, waxes, STEAROWET®, boric acid, sodium benzoate, sodium acetate, sodium chloride, leucine, a polyethylene glycol (e.g., PEG-4000) or a methoxypolyethylene glycol such as CARBOWAX™, sodium oleate, sodium benzoate, glyceryl behenate, polyethylene glycol, magnesium or sodium lauryl sulfate, colloidal silica such as SYLOID™, CAB-O-SIL®, a starch such as corn starch, silicone oil, a surfactant, and the like.

Plasticizers include compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, triethyl cellulose and triacetin. Plasticizers can also function as dispersing agents or wetting agents.

Solubilizers include compounds such as triacetin, triethyl citrate, ethyl oleate, ethyl caprylate, sodium lauryl sulfate, sodium doccusate, vitamin E TPGS, dimethylacetamide, N-methylpyrrolidone, N-hydroxyethylpyrrolidone, polyvinylpyrrolidone, hydroxypropylmethyl cellulose, hydroxypropyl cyclodextrins, ethanol, n-butanol, isopropyl alcohol, cholesterol, bile salts, polyethylene glycol 200-600, glycofurol, transcutol, propylene glycol, and dimethyl isosorbide and the like.

Stabilizers include compounds such as any antioxidation agents, buffers, acids, preservatives and the like. Exemplary stabilizers include L-arginine hydrochloride, tromethamine, albumin (human), citric acid, benzyl alcohol, phenol, disodium biphosphate dehydrate, propylene glycol, metacresol or m-cresol, zinc acetate, poly sorbate-20 or TWEEN® 20, or trometamol.

Suspending agents include compounds such as polyvinylpyrrolidone, e.g., polyvinylpyrrolidone K12, polyvinylpyrrolidone K17, polyvinylpyrrolidone K25, or polyvinylpyrrolidone K30, vinyl pyrrolidone/vinyl acetate copolymer (S630), polyethylene glycol, e.g., the polyethylene glycol can have a molecular weight of about 300 to about 6000, or about 3350 to about 4000, or about 7000 to about 5400, sodium carboxymethylcellulose, methylcellulose, hydroxypropylmethylcellulose, hydroxymethylcellulose acetate stearate, polysorbate-80, hydroxyethylcellulose, sodium alginate, gums, such as, e.g., gum tragacanth and gum acacia, guar gum, xanthans, including xanthan gum, sugars, cellulosics, such as, e.g., sodium carboxymethylcellulose, methylcellulose, sodium carboxymethylcellulose, hydroxypropylmethylcellulose, hydroxyethylcellulose, polysorbate-80, sodium alginate, polyethoxylated sorbitan monolaurate, polyethoxylated sorbitan monolaurate, povidone and the like.

Surfactants include compounds such as sodium lauryl sulfate, sodium docusate, Tween 60 or 80, triacetin, vitamin E TPGS, sorbitan monooleate, polyoxyethylene sorbitan monooleate, polysorbates, polaxomers, bile salts, glyceryl monostearate, copolymers of ethylene oxide and propylene oxide, e.g., PLURONIC® (BASF), and the like. Additional surfactants include polyoxyethylene fatty acid glycerides and vegetable oils, e.g., polyoxyethylene (60) hydrogenated castor oil, and polyoxyethylene alkyl ethers and alkylphenyl ethers, e.g., octoxynol 10, octoxynol 40. Sometimes, surfactants is included to enhance physical stability or for other purposes.

Viscosity enhancing agents include, e.g., methyl cellulose, xanthan gum, carboxymethyl cellulose, hydroxypropyl cellulose, hydroxypropylmethyl cellulose, hydroxypropylmethyl cellulose acetate stearate, hydroxypropylmethyl cellulose phthalate, carbomer, polyvinyl alcohol, alginates, acacia, chitosans and combinations thereof.

Wetting agents include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium docusate, sodium oleate, sodium lauryl sulfate, sodium doccusate, triacetin, Tween 80, vitamin E TPGS, ammonium salts and the like.

The pharmaceutical compositions for the administration of the combinations of compounds can be conveniently presented in dosage unit form and can be prepared by any of the methods well known in the art of pharmacy. The pharmaceutical compositions can be, for example, prepared by uniformly and intimately bringing the compounds provided herein into association with a liquid carrier, a finely divided solid carrier or both, and then, if necessary, shaping the product into the desired formulation. In the pharmaceutical composition, each compound of the combination provided herein is included in an amount sufficient to produce the desired therapeutic effect. For example, pharmaceutical compositions of the present technology may take a form suitable for virtually any mode of administration, including, for example, topical, ocular, oral, buccal, systemic, nasal, injection, infusion, transdermal, rectal, and vaginal, or a form suitable for administration by inhalation or insufflation.

For topical administration, the combination of compounds can be formulated as solutions, gels, ointments, creams, suspensions, etc., as is well-known in the art.

Systemic formulations include those designed for administration by injection (e.g., subcutaneous, intravenous, infusion, intramuscular, intrathecal, or intraperitoneal injection) as well as those designed for transdermal, transmucosal, oral, or pulmonary administration.

Useful injectable preparations include sterile suspensions, solutions, or emulsions of the compounds provided herein in aqueous or oily vehicles. The compositions may also contain formulating agents, such as suspending, stabilizing, and/or dispersing agents. The formulations for injection can be presented in unit dosage form, e.g., in ampules or in multidose containers, and may contain added preservatives.

Alternatively, the injectable formulation can be provided in powder form for reconstitution with a suitable vehicle, including but not limited to sterile pyrogen free water, buffer, and dextrose solution, before use. To this end, the combination of compounds provided herein can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings.

Compositions intended for oral use can be prepared according to any method known to the art for the manufacture of pharmaceutical compositions, and such compositions may contain one or more agents selected from the group consisting of sweetening agents, flavoring agents, coloring agents, and preserving agents in order to provide pharmaceutically elegant and palatable preparations. Tablets contain the combination of compounds provided herein in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They may also be coated by the techniques well known to the skilled artisan. The pharmaceutical compositions of the present technology may also be in the form of oil-in-water emulsions.

Liquid preparations for oral administration may take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations may also contain buffer salts, preservatives, flavoring, coloring, and sweetening agents as appropriate.

In some embodiments, one or more compositions disclosed herein are contained in a kit. Accordingly, in some embodiments, provided herein is a kit comprising, consisting essentially of, or consisting of one or more compositions disclosed herein and instructions for their use.

### Dosage and Dosage Formulations

In some embodiments, the compositions are administered to a subject suffering from a condition as disclosed herein, such as a human, either alone or as part of a pharmaceutically acceptable formulation, once a week, once a day, twice a day, three times a day, or four times a day, or even more frequently.

Administration of the conjugate alone or in combination with an additional therapeutic agent and compositions containing same can be effected by any method that enables delivery to the site of action. These methods include oral routes, intraduodenal routes, parenteral injection (including intravenous, subcutaneous, intramuscular, intravascular or infusion), topical, and rectal administration. Bolus doses can be used, or infusions over a period of 1, 2, 3, 4, 5, 10, 15, 20, 30, 60, 90, 120 or more minutes, or any intermediate time period can also be used, as can infusions lasting 3, 4, 5, 6, 7, 8, 9, 10, 12, 14 16, 20, 24 or more hours or lasting for 1-7 days or more. Infusions can be administered by drip, continuous infusion, infusion pump, metering pump, depot formulation, or any other suitable means.

Dosage regimens can be adjusted to provide the optimum desired response. For example, a single bolus can be administered, several divided doses can be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the subjects to be treated; each unit containing a predetermined quantity of active compound calculated to produce the desired therapeutic effect in association with the required pharmaceutical carrier. The specification for the dosage unit forms of the disclosure are dictated by and directly dependent on (a) the unique characteristics of the agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient can also be determined, as can the temporal requirements for administering each agent to provide a detectable therapeutic benefit to the patient. Accordingly, while certain dose and administration regimens are exemplified herein, these examples in no way limit the dose and administration regimen that can be provided to a patient in practicing the present disclosure.

It is to be noted that dosage values can vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present disclosure encompasses intra-patient dose-escalation as determined by the skilled artisan. Determining appropriate dosages and regimens for administration are well-known in the relevant art and would be understood to be encompassed by the skilled artisan once provided the teachings disclosed herein.

### Diagnostic Methods

In some embodiments, one or more of the methods described herein further comprise, or consists essentially of, or yet further consists of, a diagnostic step. In some instances, a sample is first obtained from a subject suspected of having a disease or condition described above. Exemplary samples include, but are not limited to, cell sample, tissue sample, tumor biopsy, liquid samples such as blood and other liquid samples of biological origin (including, but not limited to, peripheral blood, sera, plasma, ascites, urine, cerebrospinal fluid (CSF), sputum, saliva, bone marrow, synovial fluid, aqueous humor, amniotic fluid cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood. In some instances, the sample is a tumor biopsy. In some cases, the sample is a liquid sample, e.g., a blood sample. In some cases, the sample is a cell-free DNA sample.

Various methods known in the art can be utilized to determine the presence of a disease or condition described herein or to determine whether an immune response has been induced in a subject. Assessment of one or more biomarkers associated with a disease or condition, or for characterizing whether an immune response has been induced, can be performed by any appropriate method. Expression levels or abundance can be determined by direct measurement of expression at the protein or mRNA level, for example by microarray analysis, quantitative PCR analysis, or RNA sequencing analysis. Alternatively, labeled antibody systems may be used to quantify target protein abundance in the cells, followed by immunofluorescence analysis, such as FISH analysis. In one aspect, a sample is obtained to determine the presence or absence of HER2 receptors, i.e., if the cancer or tumor is HER2T.

In one aspect, the conjugate and compositions can be used to determine if a cell or tumor is HER2+ by contacting the cell or tumor sample with a conjugate that targets HER2 and detecting the binding of the conjugate to the cell or tumor. In one aspect, the conjugate is detectably labeled to facilitate detection. Binding of the conjugate to the cell or tumor indicates the cell or tumor is HER2 positive.

### Therapeutic Methods

Further disclosed herein are methods for inducing an immune response in a subject consisting essentially of, or yet further consisting administering the conjugate or composition as disclosed herein.

Further disclosed herein are methods for treating cancer in a subject in need thereof, comprising, or alternatively consisting essentially of, or yet further consisting of administering to the subject the conjugate or composition as disclosed herein.

In some embodiments, a subject is a mammal. In some embodiments, a subject is a human. In some embodiments, a subject has a condition. In some embodiments, a subject has cancer. In some embodiments, a cancer is selected from melanoma, breast cancer, prostate cancer, lung cancer, ovarian cancer, skin cancer, bladder cancer, pancreatic cancer, gastric cancer, esophageal cancer, colon cancer, glioma, cervical cancer, hepatocellular cancer, or thyroid cancer. In some embodiments, the cancer is primary or metastatic cancer. In some embodiments, the cancer is metastatic or primary ovarian cancer or breast cancer. In some embodiments, the cancer has metastasized. In one aspect, the cell or tumor expresses HER2. In another aspect, the cancer is ovarian or breast cancer. In a further aspect, the cancer is resistant to chemotherapy.

In some embodiments, administering is selected from intravenous, intra-arterial, intramuscular, intracardiac, intrathecal, subventricular, epidural, intracerebral, intracerebroventricular, sub-retinal, intravitreal, intraarticular, intraocular, intraperitoneal, intrauterine, intradermal, subcutaneous, transdermal, transmuccosal, or inhalation. In some embodiments, administering is intravenous.

The methods and compositions disclosed herein may further comprise or alternatively consist essentially of, or yet further consists of administering to the subject an anti-tumor therapy other than the conjugate. In some embodiments, anti-tumor therapy may include different cancer therapy or tumor resection. The additional therapeutic can be combined in the same composition or separately administered.

In some embodiments, the conjugate are provided to prevent the symptoms of cancer from occurring in a subject that is predisposed or does not yet display symptoms of the cancer.

In some embodiments, the conjugate or composition disclosed herein may be delivered or administered into a cavity formed by the resection of tumor tissue (i.e. intracavity delivery) or directly into a tumor prior to resection (i.e. intratumoral delivery). In some embodiments. In some embodiments, the administering is intravenous.

In some embodiments, any of the conjugate or composition disclosed herein are administered to the subject at least 1, 2, 3, 4, 5, 6, 7, 8, 9, or 10 times a day. In some embodiments, the conjugate or composition is administered to the subject at least 1, 2, 3, 4, 5, 6, 7, 8, 9, 10, 11, 12, 13, 14, 15, 16, 17, 18, 19, or 20 times a week. In some embodiments, any of the conjugate or composition is administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the conjugate or composition disclosed herein is administered to the subject at least every 1, 2, 3, 4, 5, 6, 7, 8, 9, or 10 days.

In some embodiments, the method and compositions provided herein, comprise, or alternatively consist essentially of, or yet further consist of inhibiting metastatic potential of the cancer, reduction in tumor size, a reduction in tumor burden, longer progression free survival, or longer overall survival of the subject.

In some cases, the conjugate or composition with or without the additional therapeutic agent comprise, or consists essentially of, or yet further consists of, or is used as a first-line therapy. As used herein, “first-line therapy” comprises, or consists essentially of, or yet further consists of, a primary treatment for a subject with a cancer. In some instances, the cancer is a primary cancer. In other instances, the cancer is a metastatic or recurrent cancer. In some cases, the first-line therapy comprise, or consists essentially of, or yet further consists of, chemotherapy. In other cases, the first-line treatment comprise, or consists essentially of, or yet further consists of, radiation therapy. A skilled artisan would readily understand that different first-line treatments may be applicable to different type of cancers.

In some cases, the conjugate or composition is administered as a second-line therapy, a third-line therapy, a fourth-line therapy, or a fifth-line therapy. As used herein, a second-line therapy encompasses treatments that are utilized after the primary or first-line treatment stops. They can also be used as third-line, fourth-line or fifth line therapy. A third-line therapy, a fourth-line therapy, or a fifth-line therapy encompass subsequent treatments. As indicated by the naming convention, a third-line therapy encompass a treatment course upon which a primary and second-line therapy have stopped.

In some cases, the additional therapeutic agent comprise, or consists essentially of, or yet further consists of, a salvage therapy.

In some cases, the additional therapeutic agent comprise, or consists essentially of, or yet further consists of, a palliative therapy.

In one aspect, the methods or compositions further comprise administration of an additional therapeutic agent. In some cases, the additional therapeutic agent disclosed herein comprise, or consists essentially of, or yet further consists of, a chemotherapeutic agent, an immunotherapeutic agent, a targeted therapy, radiation therapy, or a combination thereof. Illustrative additional therapeutic agents include, but are not limited to, alkylating agents such as altretamine, busulfan, carboplatin, carmustine, chlorambucil, cisplatin, cyclophosphamide, dacarbazine, lomustine, melphalan, oxalaplatin, temozolomide, or thiotepa; antimetabolites such as 5-fluorouracil (5-FU), 6-mercaptopurine (6-MP), capecitabine, cytarabine, floxuridine, fludarabine, gemcitabine, hydroxyurea, methotrexate, or pemetrexed; anthracyclines such as daunorubicin, doxorubicin, epirubicin, or idarubicin; topoisomerase I inhibitors such as topotecan or irinotecan (CPT-11); topoisomerase II inhibitors such as etoposide (VP-16), teniposide, or mitoxantrone; mitotic inhibitors such as docetaxel, estramustine, ixabepilone, paclitaxel, vinblastine, vincristine, or vinorelbine; or corticosteroids such as prednisone, methylprednisolone, or dexamethasone.

Additional therapeutic agents include for example an inhibitor of the enzyme poly ADP ribose polymerase (PARP). Exemplary PARP inhibitors include, but are not limited to, olaparib (AZD-2281, LYNPARZA®, from Astra Zeneca), rucaparib (PF-01367338, RUBRACA®, from Clovis Oncology), niraparib (MK-4827, ZEJULA®, from Tesaro), talazoparib (BMN-673, from BioMarin Pharmaceutical Inc.), veliparib (ABT-888, from Abb Vie), CK-102 (formerly CEP 9722, from Teva Pharmaceutical Industries Ltd.), E7016 (from Eisai), iniparib (BSI 201, from Sanofi), and pamiparib (BGB-290, from BeiGene).

In some cases, the additional therapeutic agent comprise, or consists essentially of, or yet further consists of, an immune checkpoint inhibitor. Exemplary checkpoint inhibitors include: PD-L1 inhibitors such as Genentech's MPDL3280A (RG7446), anti-PD-L1 monoclonal antibody MDX-1105 (BMS-936559) and BMS-935559 from Bristol-Meyer's Squibb, MSB0010718C, and AstraZeneca's MEDI4736; PD-L2 inhibitors such as GlaxoSmithKline's AMP-224 (Amplimmune), and rHIgM12B7; PD-1 inhibitors such as anti-mouse PD-1 antibody Clone J43 (Cat #BE0033-2) from BioXcell, anti-mouse PD-1 antibody Clone RMP1-14 (Cat #BE0146) from BioXcell, mouse anti-PD-1 antibody Clone EH12, Merck's MK-3475 anti-mouse PD-1 antibody (Keytruda, pembrolizumab, lambrolizumab), AnaptysBio's anti-PD-1 antibody known as ANB011, antibody MDX-1 106 (ONO-4538), Bristol-Myers Squibb's human IgG4 monoclonal antibody nivolumab (OPDIVO®, BMS-936558, MDX1106), AstraZeneca's AMP-514 and AMP-224, and Pidilizumab (CT-011) from CureTech Ltd; CTLA-4 inhibitors such as Bristol Meyers Squibb's anti-CTLA-4 antibody ipilimumab (also known as YERVOY®, MDX-010, BMS-734016 and MDX-101), anti-CTLA4 antibody clone 9H10 from Millipore, Pfizer's tremelimumab (CP-675,206, ticilimumab), and anti-CTLA4 antibody clone BNI3 from Abeam; LAG3 inhibitors such as anti-Lag-3 antibody clone eBioC9B7W (C9B7W) from eBioscience, anti-Lag3 antibody LS-B2237 from LifeSpan Biosciences, IMP321 (ImmuFact) from Immutep, anti-Lag3 antibody BMS-986016, and the LAG-3 chimeric antibody A9H12; B7-H3 inhibitors such as MGA271; KIR inhibitors such as Lirilumab (IPH2101); CD137 inhibitors such as urelumab (BMS-663513, Bristol-Myers Squibb), PF-05082566 (anti-4-1BB, PF-2566, Pfizer), or XmAb-5592 (Xencor); PS inhibitors such as Bavituximab; and inhibitors such as an antibody or fragments (e.g., a monoclonal antibody, a human, humanized, or chimeric antibody) thereof, RNAi molecules, or small molecules to TFM3, CD52, CD30, CD20, CD33, CD27, OX40, GITR, ICOS, BTLA (CD272), CD160, 2B4, LAIR1, TIGHT, LIGHT, DR3, CD226, CD2, or SLAM. In some cases, the additional therapeutic agent comprise, or consists essentially of, or yet further consists of, pembrolizumab, nivolumab, tremelimumab, or ipilimumab.

In some cases, the additional therapeutic agent comprise, or consists essentially of, or yet further consists of, an adoptive T cell transfer (ACT) therapy. In one embodiment, ACT involves identification of autologous T lymphocytes in a subject with, e.g., anti-tumor activity, expansion of the autologous T lymphocytes in vitro, and subsequent reinfusion of the expanded T lymphocytes into the subject. In another embodiment, ACT comprise, or consists essentially of, or yet further consists of, use of allogeneic T lymphocytes with, e.g., anti-tumor activity, expansion of the T lymphocytes in vitro, and subsequent infusion of the expanded allogeneic T lymphocytes into a subject in need thereof.

In some instances, the conjugate or composition is administered in combination with a radiation therapy.

### Kits

In one particular aspect, the present disclosure provides kits for performing the methods of this disclosure as well as instructions for carrying out the methods of the present disclosure. The kit comprises, or alternatively consists essentially of, or yet further consists of one or more of the conjugate or composition and instructions for use. In a further aspect, the instruction for use provide directions to conduct any of the methods disclosed herein.

The kits are useful for detecting the presence of cancer such as lung cancer in a biological sample e.g., any bodily fluid including, but not limited to, e.g., sputum, serum, plasma, lymph, cystic fluid, urine, stool, cerebrospinal fluid, acidic fluid or blood and including biopsy samples of body tissue. The test samples may also be a tumor cell, a normal cell adjacent to a tumor, a normal cell corresponding to the tumor tissue type, a blood cell, a peripheral blood lymphocyte, or combinations thereof. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kit components, (e.g., reagents) can be packaged in a suitable container. The kit can also comprise, or alternatively consist essentially of, or yet further consist of, e.g., a buffering agent, a preservative or a protein-stabilizing agent. The kit can further comprise, or alternatively consist essentially of, or yet further consist of components necessary for detecting the detectable-label, e.g., an enzyme or a substrate. The kit can also contain a control sample or a series of control samples, which can be assayed and compared to the test sample. Each component of the kit can be enclosed within an individual container and all of the various containers can be within a single package, along with instructions for interpreting the results of the assays performed using the kit. The kits of the present disclosure may contain a written product on or in the kit container. The written product describes how to use the reagents contained in the kit.

As amenable, these suggested kit components may be packaged in a manner customary for use by those of skill in the art. For example, these suggested kit components may be provided in solution or as a liquid dispersion or the like.

As is apparent to those of skill in the art, the aforementioned methods and compositions can be combined with other therapeutic composition and agents for the treatment or the disclosed diseases or conditions.

### Material and Methods

### Production of Plant Viruses/Virus-Like Particle

Any plant viruses were produced under approved USDA PPQ526 permits. 10.1401 Tobacco mosaic virus (TMV) and tobacco mild green mottle virus (TMGMV): TMV was prepared using Nicotiana benthamiana plants as described previously.⁴¹ TMGMV was obtained from BioProdex and further purified as described previously.⁴²

Physalis mottle virus-like particle (PhMV): PhMV VLPs were prepared by expressing the coat protein subunits in E. coli as previously described.⁴³

Cowpea chlorotic mottle virus (CCMV): CCMV was produced by mechanically infecting primary leaves of Vigna unguiculata, California black-eyed peas No. 5; 0.05 mg CCMV was inoculated onto carborundum-dusted leaves when plants were 12 days old. Plants were harvested at day 24 when mottling symptoms were apparent. CCMV was purified using procedures as described previously.^{44,45}

### Cowpea Mosaic Virus (CPMV) Preparation and Characterizations

Cowpea mosaic virus (CPMV) was produced in Vigna unguiculata plants by infecting each primary leave with 4 μg of CPMV. Infected leaves were harvested 12 days post-infection and purified as previously described.³⁸ Concentration of purified CPMV was determined by ultraviolet-visible (UV-Vis) at wavelength 260 nm with extinction coefficient ε260 nm=8.1 mg⁻¹ mL cm⁻¹. CPMV preparations were characterized by transmission electron microscope (TEM), dynamic light scattering (DLS), agarose gel electrophoresis, and size exclusion chromatography (SEC), as previously reported.³⁹

### Isolation of CPMV-Binding Peptides by Biopanning

The Ph.D.™-7 Phage Display Peptide Library Kit (New England Biolabs) with 10⁹ unique sequences was used for isolation of CPMV binding peptides. 5 μg of CPMV was coated into each well of Nunc Maxisorp™ flat-bottom 96-well plates and incubated overnight at 4° C. 10⁹ pfu of the phage library (in 2% (w/v) bovine serum albumin; BSA) was added after the following blocking steps to remove BSA binders. The next day, CPMV was drained off and the well blocked with 2% (w/v) BSA for 1 h at room temperature (rt) with shaking at 800 rpm. The wells were washed thrice with Tris-buffered saline with 0.1% (v/v) Tween-20 (TBST) for 1 min per wash. The phage library was then added into the well and incubated for 30 min at rt with shaking at 8 rpm. The wells were washed thrice with TBST for 1 min per wash to remove unbound phages. Concentration of Tween-20 in TBST was increased from 0.1% to 0.3% and then 0.5% for each subsequent round to increase the selection stringency. Bound phages were eluted from CPMV by incubation with 0.2 M Glycine-HCl pH 2.2 for 18 min at rt and immediately neutralized by adding 1 M Tris-HCl buffer (pH 9.1). The eluted phages were subjected for amplification and titer according to the manufacturer's protocol.

### Monoclonal Enzyme-Linked Immunosorbent Assay (ELISA)

5 μg of CPMV (in 0.1 M bicarbonate buffer, pH 8.6) was coated into each well of a Nunc Maxisorp™ flat-bottom 96-well plate and incubated overnight at 4° C. 2% (w/v) BSA was also coated to serve as negative control. Next day, the plate was blocked with 2% (w/v) BSA and incubated at rt with shaking at 800 rpm for 1 h. Subsequently, the plate was washed thrice with 0.1% TBST for 1 min. 100 μL of amplified phage from the last biopanning cycle was added into each well followed by incubation at rt with shaking at 800 rpm for 1 h. Later, the plate was washed thrice with 0.5% TBST for 5 min. 100 μL of 1:500 dilution of HRP-conjugated anti-M monoclonal antibody (Abcam ab50370) was added and incubated at rt for 1 h with shaking at 8 rpm. The plate was again washed thrice with 0.5% TBST for 5 min followed by adding 100 μL of tetramethylbenzidine (TMB) substrate (Thermo Scientific Pierce) into each well. The plate was incubated at dark for 10 min and the absorbance was measured at 370 nm using an Infinite 200 Rx plate reader (Tecan Life Sciences) with 25 flashes in 96-well flat-bottom plate mode. Monoclonal phages with at least 0.3 difference in absorbance value between CPMV and BSA were subjected for DNA sequencing (Eurofins Genomics).

### Validation of Monoclonal Phage

Binding of monoclonal phages to CPMV was validated by three separate assays:

### Dot Blot

10⁹ pfu of phage was coated on Amersham Protran 0.45 Nitrocellulose membrane (GE Healthcare) and air-dried in hood for 10 min. The membrane was blocked with 2% (w/v) BSA for 1 h at rt followed by three washing with 0.1% TBST for 1 min. 100 μg of CPMV in 0.1% TBST was added to the membrane followed by incubation at rt for 30 min with shaking at 800 rpm. Membranes were then washed thrice with 0.5% TBST for 5 min. Polyclonal anti-CPMV antibodies (1:500 dilution; custom-made through immunization of rabbits) in 2% (w/v) BSA was added to the membranes and incubated for 1 h with shaking at 800 rpm. Membranes were again washed with 0.5% TBST thrice prior to adding HRP-conjugated goat anti-rabbit antibodies (1:5000 dilution; Pacific Immunology) in 2% (w/v) BSA and incubated for 1 h at rt with shaking at 800 rpm. The membrane was then washed with 0.5% TBST prior to incubation with 1 mL of Pierce™ ECL western blotting substrate for 1 min. Lastly, membrane was exposed to Chemiluminescence mode for 40 s with ProteinSimple FluorChem®.

### Cross-Reactivity Assay

5 μgs of virus particles were coated in Nunc Maxisorp™ flat-bottom 96-well plate and performed similarly to ‘Monoclonal ELISA’ with one modification: 10⁹ pfu of phage was added into each well to test their binding to the coated virus particles.

### Titration ELISA

Two-fold serial dilutions of CPMV (64 μg, 32 μg, 16 μg, 8 μg, 4 μg, 2 μg, 1 μg, 0.5 μg, 0.25 μg, 0.125 μg, 0.0625 μg) were coated in Nunc Maxisorp™ flat-bottom 96-well plate and performed similarly to ‘Monoclonal ELISA’ with one modification: 109 pfu of phage was added into each well for binding.

### Peptide Conjugation and Characterizations

CPMV-binding peptides: CPMV-biotin (N-GWRVSEFGGGSK/biotin/-C) (SEQ ID NO: 7) and CPMV-FITC (N-GWRVSEFGGGSK/FITC/-C) (SEQ ID NO: 8) were synthesized by GenScript with 75% purity. Binding of peptide to CPMV was performed by incubating a 500-fold molar excess of peptide per CPMV; the mixture was allowed to react overnight at 4° C. Conjugated product was purified using PD MiniTrap desalting columns with Sephadex G-25 resin (Cytiva) followed by 100 kDa Amicon Ultra-0.5 centrifugal unit (dual-purification). Concentration of product was determined by Nanodrop as described above. Particle integrity was validated by DLS, and TEM as described above.

HER2 targeting ligands: CPMV-AHNP peptides: CPMV-CBP-AHNP (N-GWRVSEF-KKKKKKKKDDDD-FCDGFYACYMDVK/FITC/-C) (SEQ ID NO: 9) and CBP-AHNP (N-KKKKKKKKDDDD-FCDGFYACYMDVK/FITC/-C) (SEQ ID NO: 10) were synthesized by GenScript with 75% purity. Binding of peptide to CPMV was performed by incubating a 100-fold molar excess peptide per CPMV and incubation at 37° C. for 1 hour. Purification and quantification of the product was determined as described above.

### Validation of CBP Binding to CPMV

Binding of CPMV-peptide to CPMV was determined by five different approaches as following:

### Agarose Gel Electrophoresis

10 μg of FITC peptide conjugated CPMV was loaded onto a 1.2% (w/v) TAE agarose gel. 10 μg of CPMV and 1 μg of free CPMV-FITC peptide was loaded as control. The samples were electrophoresed at 110 V for 40 min.

### Peptide ELISA

For CPMV-CBP-FITC: Serial dilutions of CPMV (10 μg, 5 μg, 2.5 μg, 0.125 μg, 0.63 μg, 0.31 μg, 0.16 μg, 0.08 μg, 0.04, 0.02 μg) were coated in Corning 96-well solid white flat bottom polystyrene microplate. The plate was incubated overnight at 4° C. followed by blocking with 2% (w/v) BSA at rt for 1 h with shaking at 800 rpm. The plate was then washed thrice with 0.1% TBST and 0.1 μg CBP-FITC peptide was added into each well. After incubation for 30 min at rt with shaking at 800 rpm, the plate was washed thrice with 0.5% TBST for 5 min. 100 μL of distilled water was added into each well and fluorescent reading was taken with Infinite 200 Rx plate reader (Tecan Life Sciences) with excitation and emission at 496 nm and 519 nm, respectively.

For CPMV-CBP-biotin: 1 μg of CBP-biotin peptide was coated in Pierce™ Streptavidin Coated Immunoassay Plates, 96-Well (Thermo Scientific) and incubated 1 h at rt with shaking at 800 rpm. The plate was blocked with 2% (w/v) BSA followed by three washings with 0.1% TBST for 1 min per wash. 5 μg of CPMV was added for binding and incubated for 1 h at rt with shaking at 8 rpm. Then the plate was washed thrice with 0.5% TBST for 5 min each wash. 100 μL of anti-CPMV rabbit polyclonal antibodies (1:500 dilution) was added to the wells followed by incubation at rt for 1 h with shaking at 800 rpm. Following three washing steps using 0.5% TBST for 5 min, 100 μL of HRP conjugated goat anti-rabbit antibodies (1:5000 dilution; Pacific Immunology) was added to the wells and incubated at rt for 1 h with shaking at 800 rpm. Finally, the plate was washed thrice with 0.5% TBST followed by adding 100 μL of TMB substrate (Thermo Scientific Pierce) into each well. The plate was incubated in the dark for 5-10 min and the reaction was stopped with 50 μL of 2N H₂SO₄. Absorbance was measured at 450 nm using an Infinite 200 Rx plate reader (Tecan Life Sciences) with 25 flashes in 96-well flat-bottom plate mode.

### Competitive ELISA

Competitive ELISA was performed as mentioned in ‘Monoclonal ELISA’ with modifications. Two-fold serial dilutions of CPMV peptide (0.500 μg, 0.250 μg, 0.125 μg, 0.063 μg, 0.031 μg, 0.016 μg, 0.008 μg, 0.004 μg, 0.002 μg) were added separately into the well together with 10⁹ pfu of monoclonal phages. The reaction after incubation with TMB substrate was stopped with 50 μL of 2 N H₂SO₄. Colorimetric detection was carried out by measurement at 450 nm using an Infinite 200 Rx plate reader (Tecan Life Sciences) with 25 flashes in 96-well flat-bottom plate mode.

### Western Blot

10 μg of CPMV was loaded onto a NuPage™ 12% bis-tris gel (Thermo Fisher Scientific) and electrophoresed for 40 min at 200 V. Proteins were then blotted onto Amersham Protran 0.45 Nitrocellulose membrane (GE Healthcare) at 25 V for 2 h. The membrane was then blocked with 5% (w/v) BSA for 1 h at rt with gentle shaking. Subsequently, the membrane was washed thrice with 0.5% TBST for 1 min. 1 μg/mL of CPMV-biotin peptide was added to the membrane for 1 h incubation at rt. The membrane was then washed with 0.5% TBST for min. 100 μL of 1:500 dilution of HRP-conjugated anti-streptavidin monoclonal antibody (Abcam ab7403) was added and incubated at rt for 1 h. Following three washes with 0.5% TBST, the membrane was developed with DAB substrate kit, peroxidase HRP (Vector Laboratories) for 5-10 min. The DAB reagent was drained off and the bands were documented with ProteinSimple FluorChem®.

### TEM Immunogold Binding

20 μL of biotin peptide conjugated CPMV was place on formvar/carbon-coated 400 mesh copper grids (Electron Microscopy Science) for 20 min. The grid was washed once with 10 mM KP buffer, pH 7 and blocked with 1% BSA (in 0.1% Tween-20) for 30 min followed by equilibration in 0.1% BSA (in 0.1% Tween-20) for 5 min. Later, the grid was then placed onto 20 μL of streptavidin conjugated gold nanoparticle, 5 nm (Nanocs) and incubated for 2 h. Following the incubation, the grid was washed four time with 0.01% TBST (3 min each wash), twice with 10 mM KP buffer (min each wash), and thrice with distilled water (5 min each wash). Lastly, the grid was stained with 2% uranyl acetate (Fisher Scientific) for 2 min. Excess solution was blotted with filter paper. The grid was imaged with FEI Tecnai G2 Spirit transmission microscope at 80 kV.

### Flow Cytometry Analysis

RAW 264.7 macrophages and SKOV3 cells were used for cell uptake assays. First, RAW 264 cells (200,000 cells per well using v-bottom shaped 96-well plates) were incubated with 10 μg of CPMV for CPMV-CBP complexes for 1 h at 37° C. The cells were then washed twice with FACS buffer (1 mM EDTA, 25 mM HEPES, 1% (v/v) FBS and topped up with 1×PBS) and fixed with 2% (v/v) paraformaldehyde at rt for 15 min. The cells were then washed thrice with FACS buffer followed by permeabilization using FACS buffer with 0.5% (v/v) saponin. Next, cells were incubated with anti-CPMV rabbit polyclonal antibodies (1:500 dilution in FACS buffer) at 4° C. for 1 h, following by two washes with FACS buffer with 0.5% (v/v) saponin. Secondary antibody (Cy5-conjugated goat anti-rabbit IgG antibody) in FACS buffer with 0.5% (v/v) saponin and 5% (v/v) goat serum was then added to cells and incubated at 4° C. for 1 h. Finally, cells were washed twice with FACS buffer and resuspended in FACS buffer prior performing flow cytometry analysis using BD Accuri™ C6 Plus flow cytometer.

For SKOV3 cell binding studies, cells were at 4° C. throughout the assay. Cells were first washed with cold FACS buffer with 0.1 (w/v) sodium azide (FACS-SA) followed by incubation with CPMV for CPMV-CBP complexes as described above. The cells were washed twice with FACS-SA buffer and fixed, and stained as above (by omitting the permeabilization steps). Cells were analyzed using a BD Accuri™ C6 Plus flow cytometer.

### Results and Discussion

### Production and Characterization of CPMV

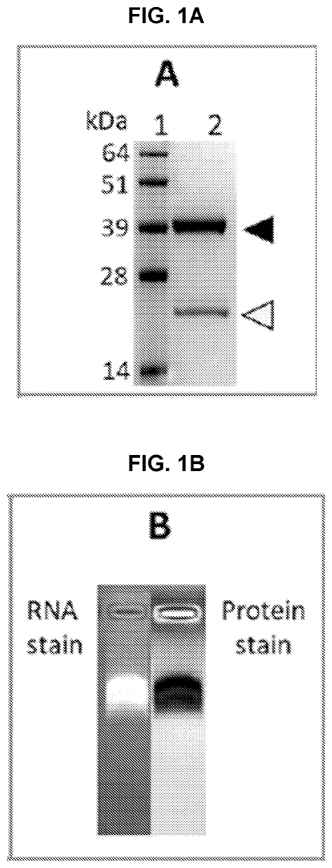

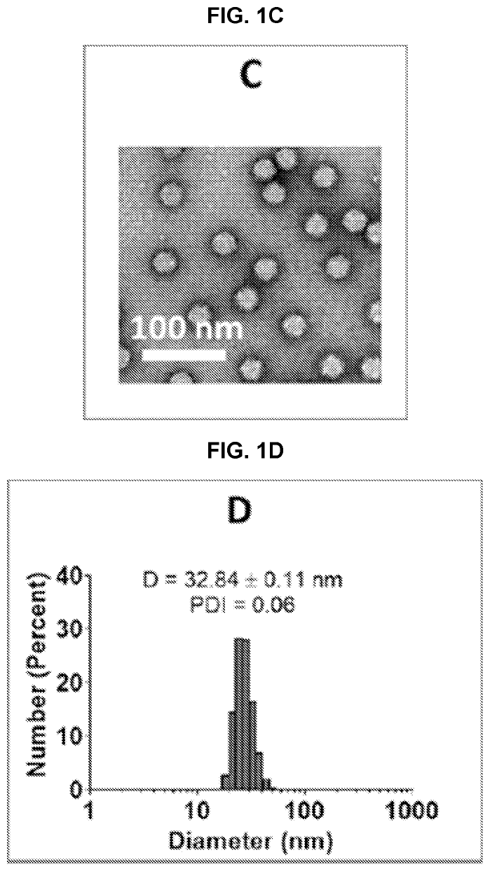

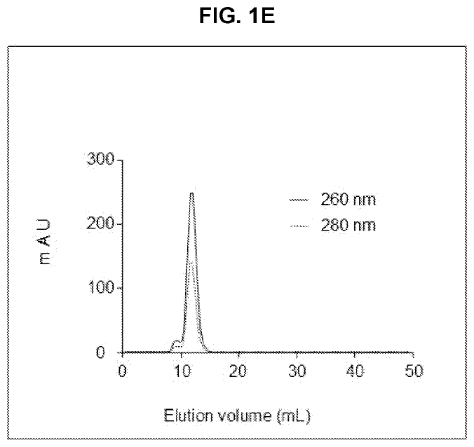

CPMV was propagated and purified from black-eyed peas and used for isolation of CPB. Purified CPMV was characterized by sodium dodecyl sulfate polyacrylamide gel electrophoresis (SDS-PAGE), native agarose gel electrophoresis, transmission electron microscope (TEM), dynamic light scattering (DLS), and size exclusion chromatography (SEC) as shown in FIG. 1. CPMV showed two distinct bands on denaturing SDS-PAGE (FIG. 1A) characteristic for the L (42 kDa; black triangle) and S (24 kDa; open triangle) proteins; protein contaminants were not detectable. The colocalization of nucleic acids and the CPMV capsids (protein) on native agarose gel (FIG. 1B) indicates that CPMV preparation yielded intact particles encapsidating their RNA molecules. TEM (FIG. 1C) and DLS (FIG. 1D) confirmed presence of monodisperse CPMV with a hydrodynamic diameter of 32 nm and low polydispersity index (PDI 0.06). SEC profiles further corroborated the presence of intact CPMV particles; aggregates or broken particles were not observed and coelution of nucleic acid (260 nm) and protein (280 nm) from the Superose6 column was evident (FIG. 1E).

### Isolation and Validation of CPMV-Binding Monoclonal Phage

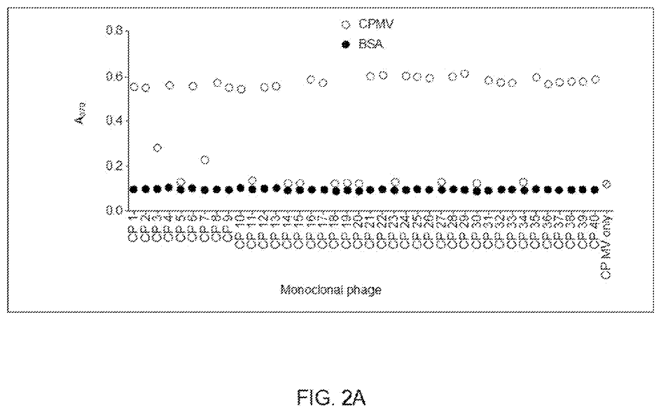

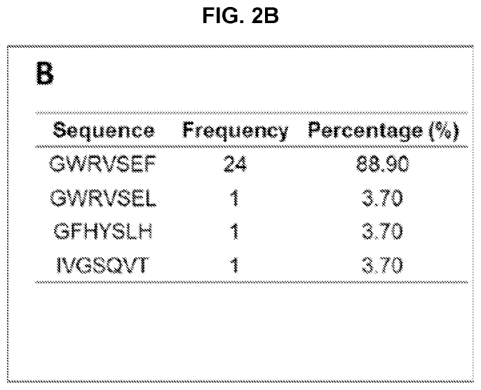

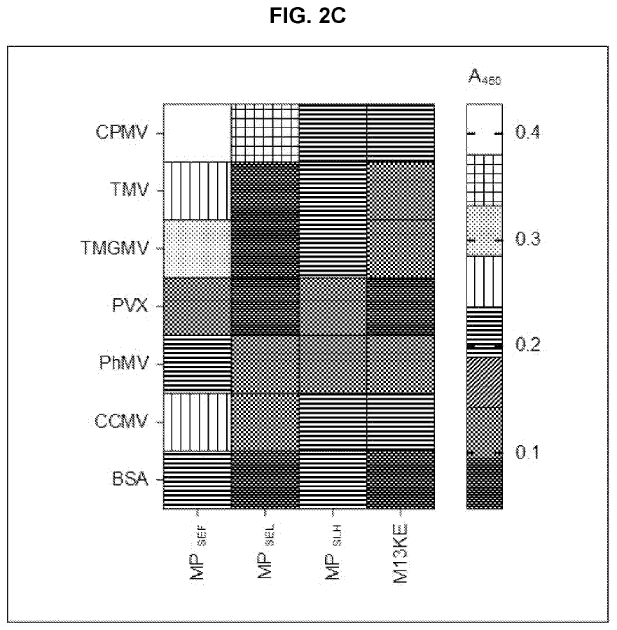

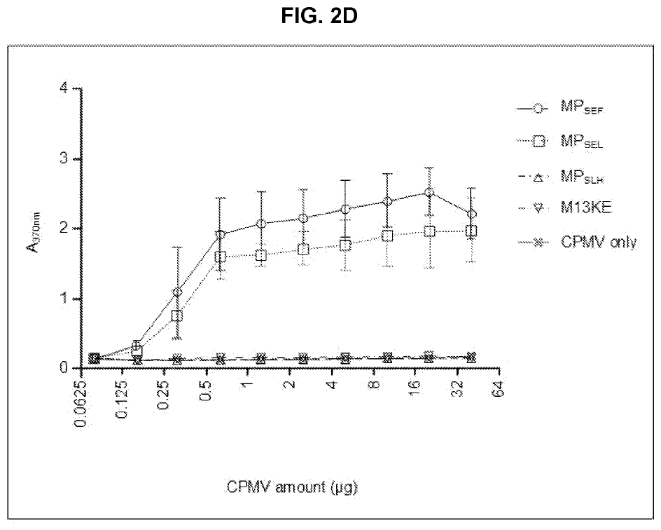

A commercial Ph.D.™-7 Phage Display Peptide Library Kit (NEB) was used to isolate potential peptide binders against CPMV. The 7-mer peptide library consisted of 1013 pfu/mL M13 phage displayed particles with 10⁹ unique peptides fused to the amino-terminus of the minor coat protein pIII of M13 phage.²² The input library was pre-incubated overnight with blocking agent (2% (w/v) bovine serum albumin; BSA) each round to remove potential BSA binders. Three rounds of ACS selections (biopanning) were performed to enrich CPMV binders by increasing the washing stringency (Tween-20 concentration was increased from 0.1% to 0.3% to 0.5% (v/v)) to remove non-specific and/or weak binders. Approximately 33-fold of enrichment was observed from third rounds of biopanning (Table 1). Forty monoclonal phages were randomly selected from the enriched phage pool to perform monoclonal enzyme-linked immunosorbent assay (ELISA) against CPMV and BSA (negative control) (FIG. 2A). Monoclonal phages yielding an absorbance difference of 0.2 units comparing CPMV- vs. BSA-binding were selected for DNA sequencing.

TABLE 1
Enrichment of CPMV-binding peptides
for every rounds of selection.

| | Input phage | Output phage | Ratio | Enrichment |
|---|---|---|---|---|
| Round | (pfu) | (pfu) | (Output/Input) | fold |
| 1 | 2 × 10¹¹ | 2 × 10⁴ | 1 × 10⁻⁷ | 1 |
| 2 | 2 × 10¹⁰ | 1 × 10⁴ | 5 × 10⁻⁷ | 5 |
| 3 | 3 × 10⁸ | 1 × 10³ | 3 × 10⁻⁶ | 33 |

Twenty seven clones were in-frame with the plasmid backbone (2 clones were either empty or out-of-frame) and sequencing yielded 4 unique sequence categories (FIG. 2B). 89% of the in-frame clones displayed the motif GWRVSEF (SEQ ID NO: 1), and 3.7% displayed highly identical motif “GWRVSEL” (SEQ ID NO: 2) suggesting consensus sequence “GWRVSE” (SEQ ID NO: 3) as the CPMV binding motif. The third unique peptide sequence “GFHYSLH” (SEQ ID NO: 4) also showed certain degree of homology with motif “GXXXSXX” (SEQ ID NO: 11), wherein X is any amino acid. The fourth identified peptide sequence “IVGSQVT” (SEQ ID NO: 5) contains several identical amino acids (V, G, S) found in “GWRVSEF” (SEQ ID NO: 1) but at different positions. Because peptide “IVGSQVT” (SEQ ID NO:5) had low similarity to the other candidate sequences, it was excluded from further study. Applicant notes that lack of homology does not reflect that peptide IVGSQVT (SEQ ID NO: 5) is not a true CMPV binder. Nevertheless, MP_{SEF} was highly enriched, covering almost 90% of the monoclonal phages binding to CPMV. Hereafter, monoclonal phage with peptide sequence GWRVSEF (SEQ ID NO: 1), GWRVSEL (SEQ ID NO: 3), and GFHYSLH (SEQ ID NO: 4) were designated as MP_{SEF}, MP_{SEL}, and MP_{SLH}, respectively.

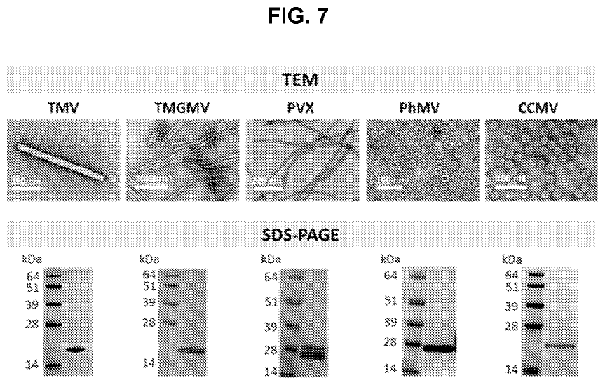

Cross-reactivity of monoclonal phages (MP_{SEF}, MP_{SEL}, and MP_{SLH}) was tested comparing their binding to CPMV vs. other non-related plant viruses, specifically tobacco mosaic virus (TMV) and tobacco mild green mosaic virus (TMGMV), both of which are 300×18 nm tubular plant viruses; potato virus X (PVX) a 515×13 filamentous plant virus; and physalis mottle virus (PhMV) and cowpea chlorotic mottle virus (CCMV), both form—like CPMV—30 nm-sized icosahedrons. It should be noted that PhMV was expressed in E. coli and used as a virus-like particle (VLP); all other plant viruses were produced in plants as plant viruses containing their genome. The methods for virus production and characterization (SDS-PAGE and TEM) are detailed in the FIG. 7. Binding assays revealed high binding of MP_{SEF} and MP_{SEL} to CPMV with MP_{SEF} exhibiting stronger binding as indicated by higher absorbance intensity read-out (FIG. 2C). Importantly, MP_{SEF} and MP_{SEL} phages demonstrated specificity toward CPMV with negligible or no interactions with any other viruses tested. MP_{SLH} in contrary showed no binding to any of the plant viruses tested—not even to CPMV or the empty M13 phage (M13KE). Enrichment of this non-specific false-positive binder might be due to its binding to one of the components used in the biopanning process and/or failure to be removed during the washing step. Presence of non-target binders in enriched pool of phages is a common observation and hence downstream verification is critical.²³ Therefore, Applicant concluded that the motif “GWRVSEF/L” (SEQ ID Nos. 1 and 2) is the CPMV-specific motif with phenylalanine (F) at the seventh position being stronger than leucine (L). The three monoclonal phages (MP_{SEF}, MP_{SEL}, MP_{SLH}) were then used for titration ELISA across a range of CPMV concentration (FIG. 2D). The result is in congruent with FIG. 2C where both the MP_{SEF}, and MP_{SEL} bound to CPMV, with MP_{SEF} showing higher binding affinity. MP_{SLH} showed no binding to CPMV.

Lastly, binding of MP_{SEF}, which showed the highest affinity to CPMV, was further assessed using a dot blot. In this approach the polystyrene surface is replaced with a nitrocellulose membrane, and therefore this allowed to rule out the possibility of false positive binding due to adsorption to polystyrene. This is important, because several studies have reported polystyrene binders within phage clones (also known as plastic binders). These target unrelated peptides (TUPs) are usually rich in hydrophobic amino acids to interact with the benzene ring of polystyrene.^{24,25} Enrichment of these TUPs would lead to isolation of false positive clones. However, FIG. 2E confirms the isolated peptide is not a TUP, because MP_{SEF} bound to CPMV using the dot blot technique and in the absence of polystyrene.

### Assessment of Synthetic CPMV-Binding Peptide (CBP) Binding to CPMV

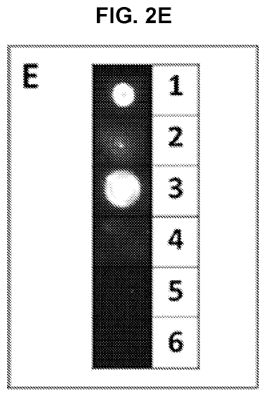

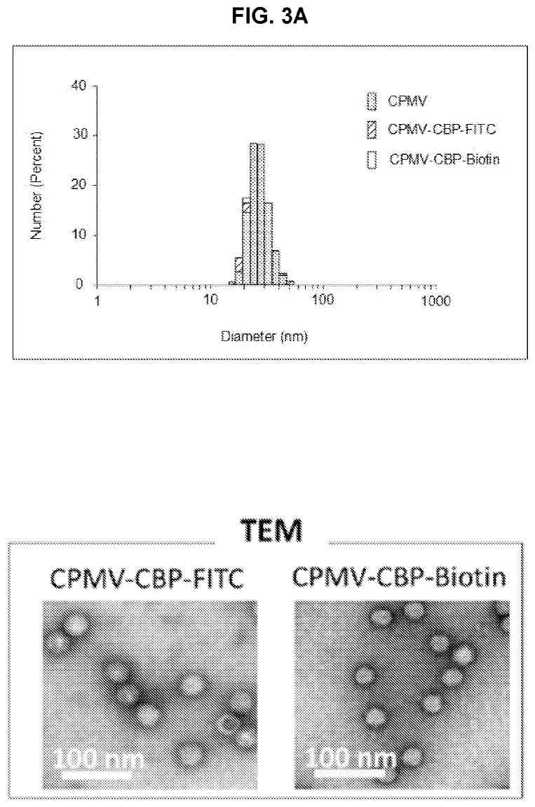

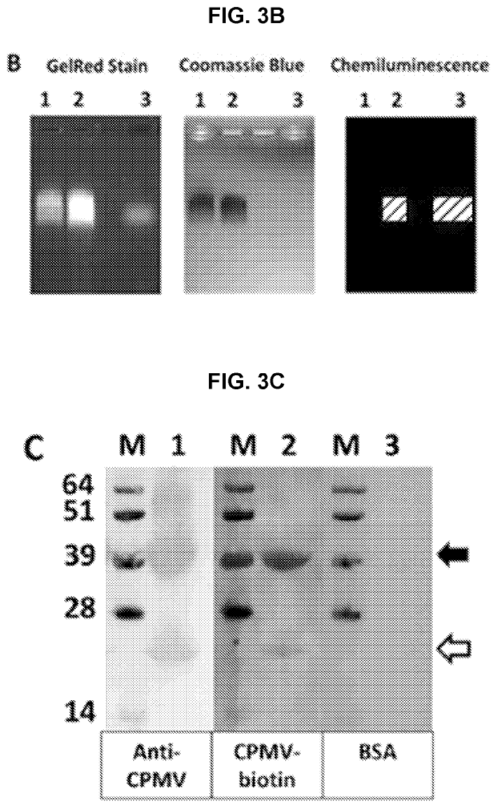

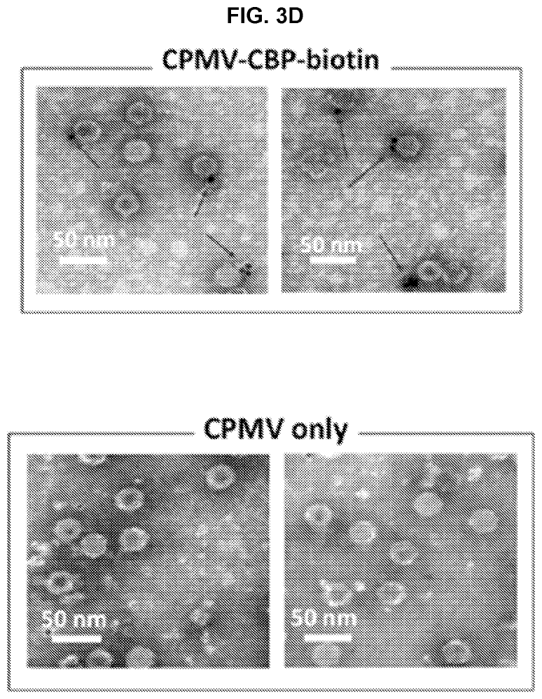

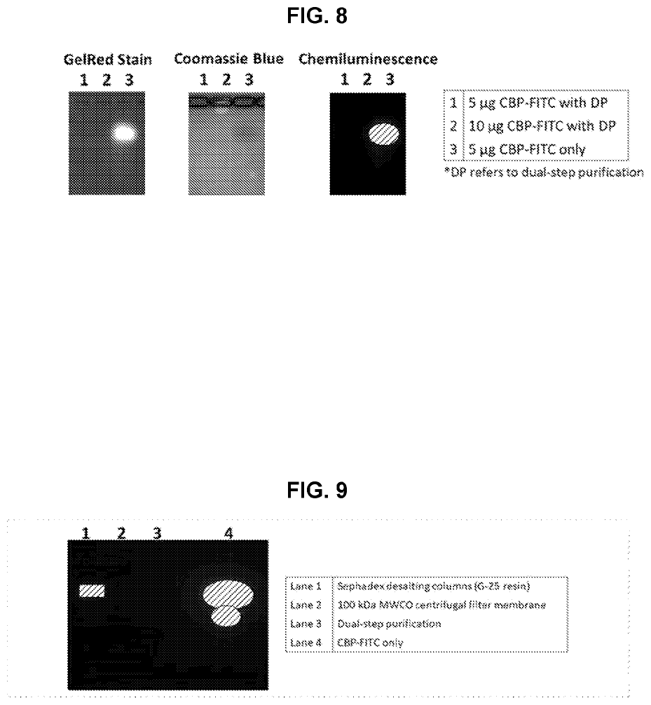

The peptide sequence GWRVSEF (SEQ ID NO: 1) from the MP_{SEF} phage was synthesized with two separate modifications at the C-terminus (C-terminal modifications were chosen, because the N-terminus is exposed and free when displayed on the M13 phage): CBP-biotin and CBP-fluorescein isothiocyanate (CBP-FITC) were synthesized to assay the interactions of the synthetic CBP peptides with CPMV. First, CBP-FITC or CBP-biotin binding to CPMV was tested by incubating the peptide at 500-molar excess in 0.1 M potassium phosphate (KP) buffer (pH 7) overnight at 4° C. This was followed by sequential purification using Sephadex desalting columns (G-25 resin) and 100 kDa molecular weight cut-off centrifugal filter membrane to remove excess peptides from the resulting CPMV-CBP-FITC complex or CPMV-CBP-biotin complex. The dual-purification was found to be efficient in removing free CBP-FITC, as free CBP-FITC could not be detected after purification as shown in FIGS. 8 and 9. CPMV-CBP-FITC and CPMV-CBP-biotin maintained stable and intact upon complexation with either peptide (CBP-CBP-FITC or CBP-CBP-biotin) as confirmed by DLS and TEM micrographs (FIG. 3A). The fluorescent construct CPMV-CBP-FITC was analyzed by native agarose gel electrophoresis and imaging under UV light followed by nucleic acid and protein staining indicates colocalization of the FITC label (chemiluminescence panel, Lane 2), nucleic acids (GelRed stain panel, Lane 2), and protein (Coomassie blue panel, Lane 2; FIG. 3B). Binding of CBP-FITC to CPMV did not alter its electrophoretic mobility. Using UV/visible spectroscopy and the CPMV and FITC-specific extinction coefficient, it was estimated that approximately 6 CBP-FITC peptides were per each CPMV particle (Table 2).

TABLE 2
Calculation of number of CBP-FITC
peptides displayed on one CPMV.

| Wavelength (nm) | Absorbance after minus blank |
|---|---|
| 260 | 0.168500185 |
| 495 | 0.015399999 |

$$\frac{Mole of CBP - FITC peptide}{Mole of CPMV} = \frac{A 495 CBP - FITC}{ε FITC * CPMV concentration ( M )} * Dilution factor$$

$$Molecular weight of CPMV = 5.6 \times {10}^{- 6} g / mol$$

$$Extinction coefficient of FITC = 68000 {M}^{- 1} {cm}^{- 1}$$

To assay the CPMV-CBP-biotin complex a modified western blot and TEM immunogold staining were used. For the modified western blot, CPMV was denatured and the coat proteins were separated by SDS-PAGE and the transferred to a nitrocellulose membrane. The coat proteins were then exposed to CBP-biotin followed by staining the biotin labels using a streptavidin HRP conjugate. Data confirmed that CBP-biotin peptides were indeed bound to the CPMV coat proteins, and binding was observed to the S and L protein (FIG. 3C). This indicated that CBP recognize the native CPMV structure as well as its denatured coat proteins. Lastly, display of CBP-biotin on CPMV was validated by transmission electron microscopy (TEM) and immunogold staining using streptavidin probes conjugated with 5 nm-sized gold nanoparticles. The micrographs revealed the immunogold staining only on CPMV-CBP-biotin (pointed by red arrows in FIG. 3D) and non-specific binding to native CPMV was not observed. Also, the micrographs demonstrate the CPMV maintained their structural integrity after attachment by CBP-biotin.

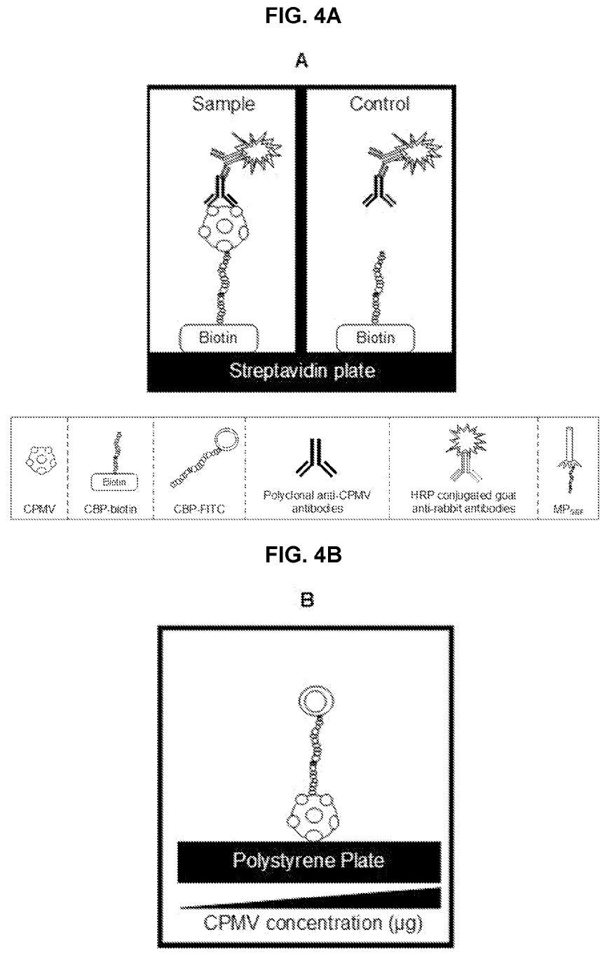

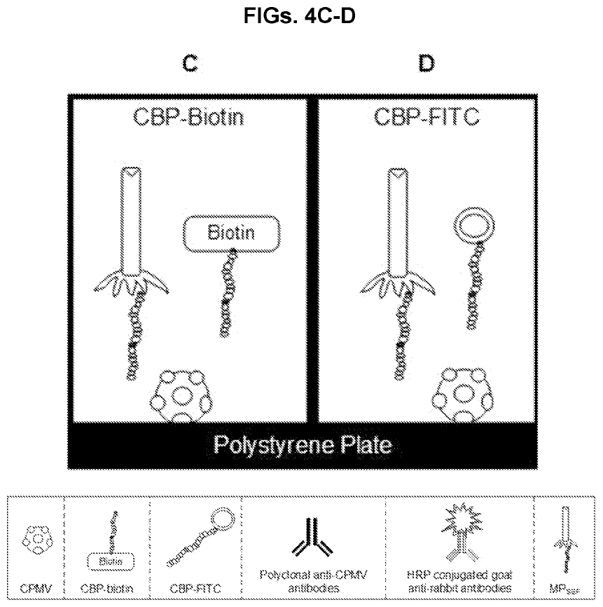

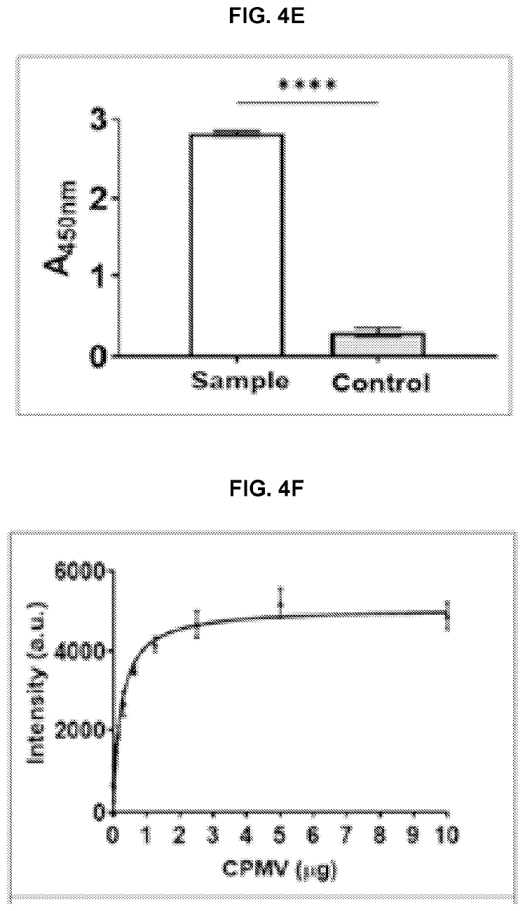

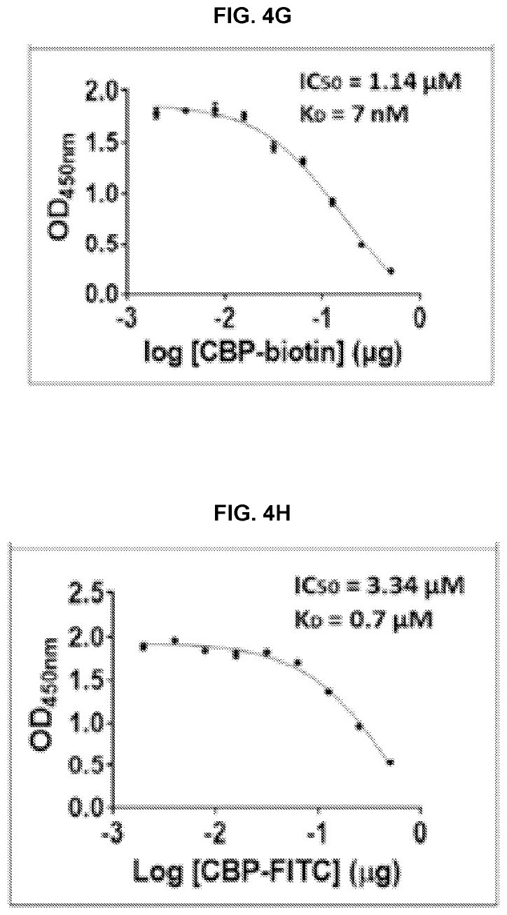

Next, Applicant performed a series of enzyme-linked immunosorbent assays (ELISAs) using the CBP-FITC and CBP-biotin to further probe their binding affinity to CPMV. CBP-biotin was coated onto a streptavidin-functionalized plate, this was followed by incubation with CPMV (CPMV was omitted for the control) (FIG. 4E). Both sample (with CPMV) and control (without CPMV) were then probed with a rabbit anti-CPMV polyclonal antibody followed by a goat anti-rabbit secondary antibody conjugated with HRP. Data are consistent with CPMV binding to CBP-biotin (unpaired t-test, p<0.0001 vs. control) (FIG. 4E). Next, a titration ELISA was performed using CPMV-CBP-FITC at serial dilutions (0 to 10 μg of CPMV-CBP-FITC) probed against CBP-FITC coated plates. Fluorescent intensity of bound CPMV-CBP-FITC was recorded and showed a typical dose-response curve with saturation reached over the concentrations tested (FIG. 4F).

To determine the dissociation constant K_{D}, competition binding ELISAs were set up. Here binding of synthetic CBPs (used in a dilution series) to CPMV was competed against MP_{SEF} (used as constant amount). Phage MP_{SEF} was detected by HRP-conjugated anti-M13 monoclonal antibody and measured at OD_{450 nm}. CPMV bound MP_{SEF} (OD_{450 nm}) is inversely proportionate to the amount of peptide loaded for competition. The amount of MP_{SEF} binding to CPMV reduced with the increasing amount of synthetic peptides, subsequently yielded lower absorbance value (FIG. 4G and FIG. 4H). The half maximal inhibition concentration (IC₅₀) of both CBP-biotin and CBP-FITC tabulated were 0.17 μg (1.14 μM) and 0.56 μg (3.34 μM), respectively. CBP-biotin demonstrated a significantly lower dissociation constant KD (˜7 nM) compared to CBP-FITC (˜0.7 μM). Different modes of peptide modification resulted different IC50 and KD values despite having the exact same sequence. The FITC fluorophore modification may impair protein binding of the CBP ligand; as has been observed with other systems and can be explained by stereo-chemical hindrance.²⁶ The difference in charge of CBP-FITC vs. CBP-biotin may also contribute to the differences in binding affinity.²⁷ CBP-FITC is negatively charged and therefore electrostatic repulsion between CBP-FITC and CPMV may weaken the interaction; in contrast biotin does not carry a charge. Hence, caution needs to be exercised when choosing the type of peptide modification to avoid the detrimental effect on the binding affinity.

### HER2-Targeted CPMV by Use of Dual-Functional CBP-AHNP

To demonstrate the applicability of the CBP to label and introduce an active ingredient to CPMV, Applicant choses to label CPMV with a receptor targeting ligand. Specifically Applicant made use of the anti-HER2/neu-peptide (AHNP) peptide with demonstrated specificity for the human growth factor receptor 2 (HER2).²⁸ AHNP is a cyclic peptide (FCDGFYACYMDV) the peptide is cyclized via the Cys side chains, highlighted as C) derived from monoclonal antibody trastuzumab and it has been shown that this minimal sequence binds to p185HER2/neu with high affinity (K_{D}=300 nM).²⁹ HER2 is transmembrane protein tyrosine kinase receptor that has linked to human cancers.³⁰

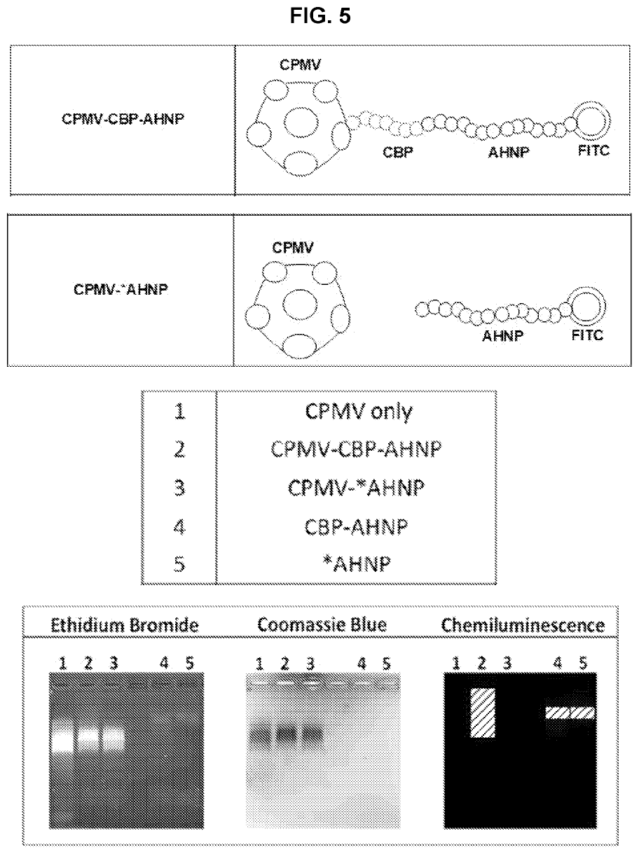

The dual functional CBP-AHNP peptide was synthesized by fusing AHNP to the C-terminus of the CBP domain. A control peptide (*AHNP) without the CBP domain was also synthesized; both peptides were modified with a FITC label at their C terminus. CPMV-CBP-AHNP and controls were characterized by native agarose gel electrophoresis as described above. Co-localization of the fluorescent signal (from the FITC-labeled CBP-AHNP peptide) with the RNA and protein band from CPMV indicates successful labeling of CPMV with CBP-AHNP, FIG. 5B, Lane 2). No fluorescent band was observed when *AHNP was incubated with CPMV (FIG. 5B, Chemiluminescence panel, Lane 3), demonstrating there is no non-specific interaction between *AHNP and CPMV.

Next cell uptake was assayed of CPMV-CBP-AHNP vs. CPMV using a HER2+ ovarian cancer cell line, SKOV3. Applicant also assayed cell uptake using murine macrophage RAW 264.7 cells to monitor non-specific vs. targeted uptake in immune cells vs. cancer cells. For these experiments, CPMV samples were prepared by incubating CPMV with a 100-molar excess of AHNP peptides (CBP-AHNP or *AHNP) in 0.1 M KP buffer; excess peptides were removed by dual-purification as described above. The FITC-label allows to track the peptides and CPMV was detected be immunostaining using rabbit anti-CPMV polyclonal antibodies followed by staining with a Cy5-labeled goat anti-rabbit secondary antibody. The Cy5 label is spectrally distinct from FITC and therefore the CPMV carrier and peptide ligand (FITC-labeled CBP-AHNP) can be detected simultaneously.

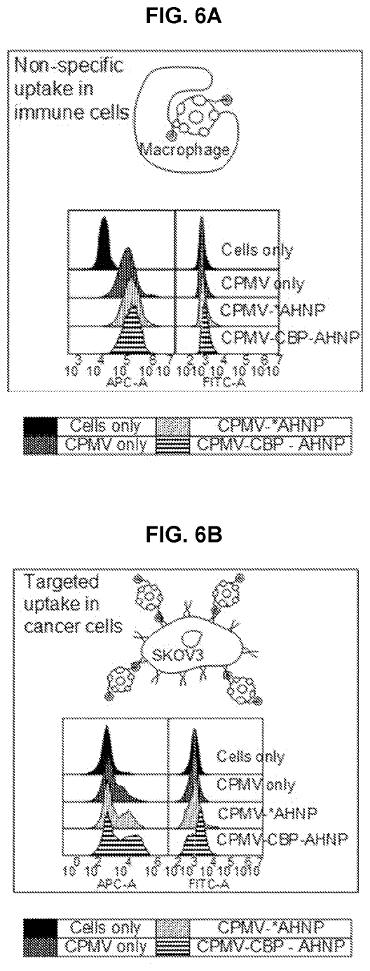

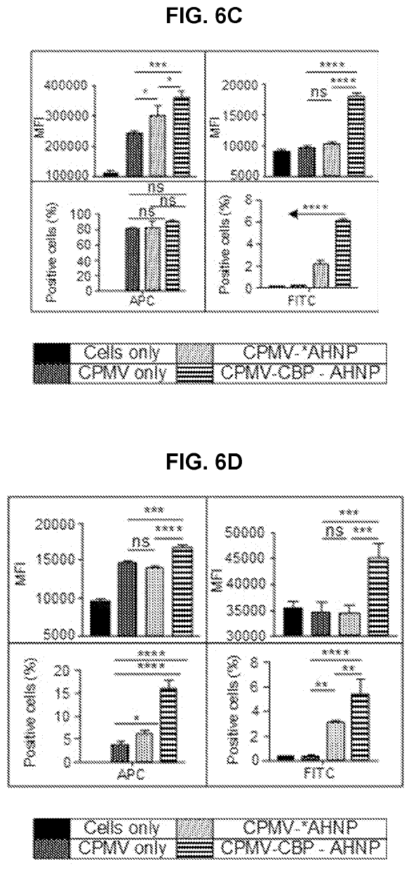

Macrophages were used to monitor non-specific phagocytosis—both, targeted and non-targeted CPMV nanoparticles are expected to be cleared by phagocytic innate immune cells.^{31,32,33} And indeed, flow cytometry indicates that CPMV, CPMV*AHNP and CPMV-CBP-AHNP were taken up by the RAW 264.7 cells; differences in cell uptake were not apparent (FIG. 6A, APC channel). Differences were noted when analyzing the FITC channel, monitoring uptake of the peptide into cells: CPMV-CBP-AHNP showed a significantly higher percentage of FITC positive cells, about 3-fold higher than CPMV-*AHNP (p<0.0001) and 23-fold higher than CPMV only (p<0.0001) (FIG. 6A, FITC channel). This indicates that the AHNP peptide is taken up by cells; but the CPMV nanoparticle is more effectively taken up.

Next Applicant assayed targeted uptake using the HER2+ SKOV3 ovarian cancer cell line.³⁶ Cell binding assays show that CPMV-CBP-AHNP had significantly greater interactions with the HER2+ cancer cell line compared to the control samples, with a ˜4.2-fold and ˜10-fold increase in %-positive cells compared to non-targeted CPMV controls (FIG. 6B). The background cell binding of CPMV and CPMV*AHNP may be explained by non-specific nanoparticle-cell interactions or the fact that CPMV interacts with mammalian cells via surface expressed vimentin.³⁷

### CONCLUSION

Applicant report the isolation of a CPMV-binding peptide (CBP) that binds CPMV with high selectivity and affinity; cross-reactivity against other plant viruses was not observed. Biopanning resulted in isolation of four lead candidates and characterization assays to evaluate the peptide-to-CPMV binding concluded that the motif “GWRVSEF/L” is the CPMV-specific motif with phenylalanine (F) at the seventh position being stronger than leucine (L). GWRVSEF tagged with biotin, FITC or HER2-specific targeting peptide ANHP were synthesized and Applicant demonstrated that the active ingredient could be displayed on CPMV using the CBP strategy. CBP binds to intact virions and was found bound to the S and L protein denatured and bound to nitrocellulose membranes. The CBP functionalization strategy offers a new avenue for CPMV nanoparticle functionalization and should offer a versatile tool to add active ingredient that otherwise may be difficult to conjugate or display. The concepts disclosed herein are applied to display peptide epitopes for vaccination or therapeutics for drug delivery or immunotherapy.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

Unless otherwise defined, all technical and scientific terms used herein have the same meaning as commonly understood by one of ordinary skill in the art to which this disclosure belongs. All nucleotide sequences provided herein are presented in the 5′ to 3′ direction.

The embodiments illustratively described herein may suitably be practiced in the absence of any element or elements, limitation or limitations, not specifically disclosed herein. Thus, for example, the terms “comprising,” “including,” containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure.

Thus, it should be understood that although the present disclosure has been specifically disclosed by specific embodiments and optional features, modification, improvement and variation of the embodiments therein herein disclosed may be resorted to by those skilled in the art, and that such modifications, improvements and variations are considered to be within the scope of this disclosure. The materials, methods, and examples provided here are representative of particular embodiments, are exemplary, and are not intended as limitations on the scope of the disclosure.

The scoped of the disclosure has been described broadly and generically herein. Each of the narrower species and subgeneric groupings falling within the generic disclosure also form part of the disclosure. This includes the generic description with a proviso or negative limitation removing any subject matter from the genus, regardless of whether or not the excised material is specifically recited herein.

In addition, where features or aspects of the disclosure are described in terms of Markush groups, those skilled in the art will recognize that embodiments of the disclosure may also thereby be described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Sequence IDs
SEQ ID NO: 1:
GWRVSEF
(Binding peptide)
SEQ ID NO: 2:
GWRVSEL
(Binding peptide)
SEQ ID NO: 3:
GWRVSE
(Binding peptide)
SEQ ID NO: 4
GFHYSLH
(Binding peptide)
SEQ ID NO: 5:
IVGSQVT
(Binding peptide)
SEQ ID NO: 6:
FCDGFYACYMDV
(Her-2 targeting ligand)
SEQ ID NO: 7:
GWRVSEFGGGSK/biotin
(CPMV-biotin)
SEQ ID NO: 8:
GWRVSEFGGGSK/FITC
(CPMV-FITC)
SEQ ID NO: 9:
GWRVSEF-KKKKKKKKDDDD-FCDGFYACYMDVK/FITC
(CPMV-CBP-AHNP)
SEQ ID NO: 10:
N-KKKKKKKKDDDD-FCDGFYACYMDVK/FITC
(CBP-AHNP)
SEQ ID NO: 11:
GXXXSXX
(Generic binding motif)

### REFERENCES

(1) Lomonossoff, G. P. Cowpea Mosaic Virus. In Encyclopedia of Virology; Elsevier Ltd, 2008; pp 569-574.
(2) Sainsbury, F.; Cañizares, M. C.; Lomonossoff, G. P. Cowpea Mosaic Virus: The Plant Virus-Based Biotechnology Workhorse. Annu. Rev. Phytopathol. 2010, 48, 437-455.
(3) CHANT, S. R. VIRUSES OF COWPEA, VIGNA UNGUICULATA L. (WALP.), IN NIGERIA. Ann. Appl. Biol. 1959, 47, 565-572.
(4) Nkanga, C. I.; Steinmetz, N. F. The Pharmacology of Plant Virus Nanoparticles. Virology 2021, 556, 39-61.
(5) Chung, Y. H.; Cai, H.; Steinmetz, N. F. Viral Nanoparticles for Drug Delivery, Imaging, Immunotherapy, and Theranostic Applications. Adv. Drug Deliv. Rev. 2020, 156, 214-235.
(6) Lizotte, P. H.; Wen, A. M.; Sheen, M. R.; Fields, J.; Rojanasopondist, P.; Steinmetz, N. F.; Fiering, S. In Situ Vaccination with Cowpea Mosaic Virus Nanoparticles Suppresses Metastatic Cancer. Nat. Nanotechnol. 2016, 11, 295-303.
(7) Wang, C.; Fiering, S. N.; Steinmetz, N. F. Cowpea Mosaic Virus Promotes Anti-Tumor Activity and Immune Memory in a Mouse Ovarian Tumor Model. Adv. Ther. 2019, 2, 1900003.
(8) Patel, B. K.; Wang, C.; Lorens, B.; Levine, A. D.; Steinmetz, N. F.; Shukla, S. Cowpea Mosaic Virus (CPMV)-Based Cancer Testis Antigen NY-ESO-1 Vaccine Elicits an Antigen-Specific Cytotoxic T Cell Response. ACS Appl. Bio Mater. 2020, 3, 4179-4187.
(9) Beatty, P. H.; Lewis, J. D. Cowpea Mosaic Virus Nanoparticles for Cancer Imaging and Therapy. Advanced Drug Delivery Reviews. Elsevier B. V. May 1, 2019, pp 130-144.
(10) Shukla, S.; Wang, C.; Beiss, V.; Cai, H.; Washington, T.; Murray, A. A.; Gong, X.; Zhao, Z.; Masarapu, H.; Zlotnick, A.; Fiering, S.; Steinmetz, N. F. The Unique Potency of Cowpea Mosaic Virus (CPMV): In Situ Cancer Vaccine. Biomater. Sci. 2020, 8, 5489-5503.
(11) Usha, R.; Rohll, J. B.; Spall, V. E.; Shanks, M.; Maule, A. J.; Johnson, J. E.; Lomonossofft, G. P. Expression of an Animal Virus Antigenic Site on the Surface of a Plant Virus Particle. Virology 1993, 197, 366-374.
(12) Madi, M.; Mioulet, V.; King, D. P.; Lomonossoff, G. P.; Montague, N. P. Development of a Non-Infectious Encapsidated Positive Control RNA for Molecular Assays to Detect Foot-and-Mouth Disease Virus. J. Virol. Methods 2015, 220, 27-34.
(13) Yildiz, I.; Lee, K. L.; Chen, K.; Shukla, S.; Steinmetz, N. F. Infusion of Imaging and Therapeutic Molecules into the Plant Virus-Based Carrier Cowpea Mosaic Virus: Cargo-Loading and Delivery. J. Control. Release 2013, 172, 568-578.
(14) Zampieri, R.; Brozzetti, A.; Pericolini, E.; Bartoloni, E.; Gabrielli, E.; Roselletti, E.; Lomonosoff, G.; Meshcheriakova, Y.; Santi, L.; Imperatori, F.; Merlin, M.; Tinazzi, E.; Dotta, F.; Nigi, L.; Sebastiani, G.; Pezzotti, M.; Falorni, A.; Avesani, L. Prevention and Treatment of Autoimmune Diseases with Plant Virus Nanoparticles. Sci. Adv. 2020, 6, eaaz0295.
(15) Pokorski, J. K.; Steinmetz, N. F. The Art of Engineering Viral Nanoparticles. Molecular Pharmaceutics. NIH Public Access Feb. 7, 2011, pp 29-43.
(16) Wang, Q.; Lin, T.; Tang, L.; Johnson, J. E.; Finn, M. G. Icosahedral Virus Particles as Addressable Nanoscale Building Blocks. Angew. Chemie—Int. Ed. 2002, 41, 459-462.
(17) Wen, A. M.; Shukla, S.; Saxena, P.; Aljabali, A. A. A.; Yildiz, I.; Dey, S.; Mealy, J. E.; Yang, A. C.; Evans, D. J.; Lomonossoff, G. P.; Steinmetz, N. F. Interior Engineering of a Viral Nanoparticle and Its Tumor Homing Properties. Biomacromolecules 2012, 13, 3990-4001.
(18) Porta, C.; Spall, V. E.; Findlay, K. C.; Gergerich, R. C.; Farrance, C. E.; Lomonossoff, G. P. Cowpea Mosaic Virus-Based Chimaeras: Effects of Inserted Peptides on the Phenotype, Host Range, and Transmissibility of the Modified Viruses. Virology 2003, 310, 50-63.
(19) Lomonossoff, G. P. Antigen Delivery Systems III: Use of Recombinant Plant Viruses. In Mucosal Immunology, Two-Volume Set; Elsevier Inc., 2005; pp 1061-1072.
(20) Smith, G. P. Filamentous Fusion Phage: Novel Expression Vectors That Display Cloned Antigens on the Virion Surface. Science (80-.). 1985, 228, 1315-1317.
(21) Wu, C. H.; Liu, I. J.; Lu, R. M.; Wu, H. C. Advancement and Applications of Peptide Phage Display Technology in Biomedical Science. Journal of Biomedical Science. BioMed Central Ltd. Jan. 19, 2016, pp 1-14.
(22) Ph.D.™-7 Phage Display Peptide Library Kit|NEB https://www.neb.com/products/e8100-phd-7-phage-display-peptide-library-kit#Product Information (accessed Feb. 15, 2021).
(23) Bakhshinejad, B.; Zade, H. M.; Shekarabi, H. S. Z.; Neman, S. Phage Display Biopanning and Isolation of Target-Unrelated Peptides: In Search of Nonspecific Binders Hidden in a Combinatorial Library. Amino Acids. Springer-Verlag Wien Dec. 1, 2016, pp 2699-2716.
(24) Hu, Y.-F.; Gao, X.-C.; Xu, T.-Q.; Dun, Z.; Yu, X.-L. Characterization of Seven New Polystyrene Plates Binding Peptides from a Phage-Displayed Random 12-Peptide Library. Comb. Chem. High Throughput Screen. 2016, 19, 283-289.
(25) Qiang, X.; Sun, K.; Xing, L.; Xu, Y.; Wang, H.; Zhou, Z.; Zhang, J.; Zhang, F.; Caliskan, B.; Wang, M.; Qiu, Z. Discovery of a Polystyrene Binding Peptide Isolated from Phage Display Library and Its Application in Peptide Immobilization. Sci. Rep. 2017, 7, 1-11.
(26) Sun, Y. S.; Landry, J. P.; Fei, Y. Y.; Zhu, X. D.; Luo, J. T.; Wang, X. B.; Lam, K. S. Effect of Fluorescently Labeling Protein Probes on Kinetics of Protein-Ligand Reactions. Langmuir 2008, 24, 13399-13405.
(27) Yin, L.; Wang, W.; Wang, S.; Zhang, F.; Zhang, S.; Tao, N. How Does Fluorescent Labeling Affect the Binding Kinetics of Proteins with Intact Cells? Biosens. Bioelectron. 2015, 66, 412-416.
(28) Guan, S. S.; Wu, C. T.; Chiu, C. Y.; Luo, T. Y.; Wu, J. Y.; Liao, T. Z.; Liu, S. H. Polyethylene Glycol-Conjugated HER2-Targeted Peptides as a Nuclear Imaging Probe for HER2-Overexpressed Gastric Cancer Detection in Vivo. J. Transl. Med. 2018, 16, 168.
(29) Park, B. W.; Zhang, H. T.; Wu, C.; Berezov, A.; Zhang, X.; Dua, R.; Wang, Q.; Kao, G.; O'Rourke, D. M.; Greene, M. I.; Murali, R. Rationally Designed Anti-HER2/Neu Peptide Mimetic Disables P185 (HER2/Neu) Tyrosine Kinases in Vitro and in Vivo. Nat. Biotechnol. 2000, 18, 194-198.
(30) Bermont, L.; Algros, M. P.; Baron, M. H.; Adessi, G. L. Relevance of P185 HER-2/Neu Oncoprotein Quantification in Human Primary Breast Carcinoma. Breast Cancer Res. Treat. 2000, 63, 163-169.
(31) Wen, A. M.; Le, N.; Zhou, X.; Steinmetz, N. F.; Popkin, D. L. Tropism of CPMV to Professional Antigen Presenting Cells Enables a Platform to Eliminate Chronic Infections. ACS Biomater. Sci. Eng. 2015, 1, 1050-1054.
(32) Plummer, E. M.; Manchester, M. Endocytic Uptake Pathways Utilized by CPMV Nanoparticles. Mol. Pharm. 2013, 10, 26-32.
(33) Gonzalez, M. J.; Plummer, E. M.; Rae, C. S.; Manchester, M. Interaction of Cowpea Mosaic Virus (CPMV) Nanoparticles with Antigen Presenting Cells In Vitro and In Vivo. PLoS One 2009, 4, e7981.
(34) Gustafson, H. H.; Holt-Casper, D.; Grainger, D. W.; Ghandehari, H. Nanoparticle Uptake: The Phagocyte Problem. Nano Today. Elsevier B. V. Aug. 1, 2015, pp 487-510.
(35) Murray, P. J.; Wynn, T. A. Protective and Pathogenic Functions of Macrophage Subsets. Nature Reviews Immunology. NIH Public Access November 2011, pp 723-737.
(36) Zanini, E.; Louis, L. S.; Antony, J.; Karali, E.; Okon, I. S.; McKie, A. B.; Vaughan, S.; El-Bahrawy, M.; Stebbing, J.; Recchi, C.; Gabra, H. The Tumor-Suppressor Protein OPCML Potentiates Anti-EGFR- and Anti-HER2-Targeted Therapy in HER2-Positive Ovarian and Breast Cancer. Mol. Cancer Ther. 2017, 16, 2246-2256.
(37) Koudelka, K. J.; Destito, G.; Plummer, E. M.; Trauger, S. A.; Siuzdak, G.; Manchester, M. Endothelial Targeting of Cowpea Mosaic Virus (CPMV) via Surface Vimentin. PLoS Pathog. 2009, 5.
(38) Wellink, J. Comovirus Isolation and RNA Extraction. Methods Mol. Biol. 1998, 81, 205-209.
(39) Chan, S. K.; Du, P.; Ignacio, C.; Mehta, S.; Newton, I. G.; Steinmetz, N. F. Biomimetic Virus-Like Particles as Severe Acute Respiratory Syndrome Coronavirus 2 Diagnostic Tools. ACS Nano 2020, acsnano.0c08430.
(40) Masarapu, H.; Patel, B. K.; Chariou, P. L.; Hu, H.; Gulati, N. M.; Carpenter, B. L.; Ghiladi, R. A.; Shukla, S.; Steinmetz, N. F. Physalis Mottle Virus-Like Particles as Nanocarriers for Imaging Reagents and Drugs. Biomacromolecules 2017, 18, 4141-4153.
(41) Bruckman, M. A.; Steinmetz, N. F. Chemical Modification of the Inner and Outer Surfaces of Tobacco Mosaic Virus (TMV). Methods Mol. Biol. 2014, 1108, 173-185.
(42) Chariou, P. L.; Steinmetz, N. F. Delivery of Pesticides to Plant Parasitic Nematodes Using Tobacco Mild Green Mosaic Virus as a Nanocarrier. ACS Nano 2017, 11, 4719-4730.
(43) Sastri, M.; Kekuda, R.; Gopinath, K.; Ranjith Kumar, C. T.; Jagath, J. R.; Savithri, H. S. Assembly of Physalis Mottle Virus Capsid Protein in Escherichia Coli and the Role of Amino and Carboxy Termini in the Formation of the Icosahedral Particles. J. Mol. Biol. 1997, 272, 541-552.
(44) Chan, S. K.; Du, P.; Ignacio, C.; Mehta, S.; Newton, I. G.; Steinmetz, N. F. Biomimetic Virus-Like Particles as Severe Acute Respiratory Syndrome Coronavirus 2 Diagnostic Tools. ACS Nano 2020, acsnano.0c08430.
(45) Chan, S. K.; Du, P.; Ignacio, C.; Mehta, S.; Newton, I. G.; Steinmetz, N. F. Virus-Like Particles as Positive Controls for COVID-19 RT-LAMP Diagnostic Assays. Biomacromolecules 2021.
(46) Lee, K. L.; Shukla, S.; Wu, M.; Ayat, N. R.; El Sanadi, C. E.; Wen, A. M.; Edelbrock, J. F.; Pokorski, J. K.; Commandeur, U.; Dubyak, G. R.; Steinmetz, N. F. Stealth Filaments: Polymer Chain Length and Conformation Affect the in Vivo Fate of PEGylated Potato Virus X. Acta Biomater. 2015, 19, 166-179.

1. A conjugate comprising at least one peptide selected from: N-GWRVSEL-C (SEQ ID NO: 2), N-GWRVSEF-C (SEQ ID NO: 1), N-GWRVSE-C (SEQ ID NO: 3), N-GFHYSLH-C (SEQ ID NO: 4), or N-IVGSQVT-C (SEQ ID NO: 5) conjugated to the surface of a cowpea mosaic virus (CPMV) or CMPV coat protein (CP) wherein the N-terminus of the peptide linked to the CPMV or CP surface.

2. The conjugate of claim 1, wherein the peptide comprises N-GWRVSEL-C (SEQ ID NO: 2) or N-GWRVSEF-C (SEQ ID NO: 1).

3. The conjugate of claim 1, further comprising a lysine at the C-terminus of the peptide.

4. The conjugate of claim 3, wherein the lysine is chemically modified to further comprise one or more of a peptide, FITC, a ligand, biotin, fluorophore, an anti-cancer agent, or a human epidermal growth factor receptor 2 (HER2)-specific targeting peptide ligand.

5. The conjugate of claim 1, further comprising active agents conjugated to the exterior or interior surface of the CPMV or CP by covalent coupling of the active agents to the coat proteins of the CPMV.

6. The conjugate of claim 1, further comprising an agent infused into the CPMV or CP.

7. The conjugate of claim 1, wherein the CPMV or CP is modified.

8. The conjugate of claim 7, wherein the CPMV or CP is chemically modified by a method comprising one or more of bioconjugation, targeting solvent-exposed lysine side chains using N-hydroxysuccinimidyl ester (NHS) reaction and biorthogonal click-chemistry such as copper-catalyzed azide-alkyne cycloaddition (CuAAC).

9. The conjugate of claim 1, wherein the interior of the CPMV or CP is modified by one or more of targeting cysteine side chains on the interior capsid surface or targeting surface loops.

10. The conjugate of claim 4, wherein the lysine is chemically modified to further comprise a human epidermal growth factor receptor 2 (HER2)-specific targeting peptide ligand.

11. The conjugate of claim 10, wherein the ligand comprises the peptide FCDGFYACYMDV (SEQ ID NO: 6).

12. A plurality of the conjugates of claim 1.

13. The plurality of claim 12, wherein the conjugates are the same or different from each other.

14. A composition comprising the conjugate of claim 1, and a carrier.

15. The composition of claim 14, wherein the carrier is a pharmaceutically acceptable carrier.

16. A method of inhibiting the growth of a HER-2 expressing cancer cell comprising contacting the cell with the conjugate of claim 10.

17. (canceled)

18. The method of claim 16, wherein the cell is a breast cancer cell or an ovarian cancer cell.

19. (canceled)

20. A method of treating a HER-2 expressing cancer in a subject in need thereof comprising administering an effective amount of the conjugate of claim 9 to the subject.

21. The method of claim 20, wherein the cancer is a breast cancer cell or an ovarian cancer cell.

22. The method of claim 20, wherein the cell is an ovarian cancer cell that is resistant to chemotherapy.

23. (canceled)


SEQUENCE LISTING (12 sequences)

SEQ ID NO: 1 - protein, 7 aa, Artificial Sequence
  Description of Artificial Sequence Synthetic peptide
GWRVSEF

SEQ ID NO: 2 - protein, 7 aa, Artificial Sequence
  Description of Artificial Sequence Synthetic peptide
GWRVSEL

SEQ ID NO: 3 - protein, 6 aa, Artificial Sequence
  Description of Artificial Sequence Synthetic peptide
GWRVSE

SEQ ID NO: 4 - protein, 7 aa, Artificial Sequence
  Description of Artificial Sequence Synthetic peptide
GFHYSLH

SEQ ID NO: 5 - protein, 7 aa, Artificial Sequence
  Description of Artificial Sequence Synthetic peptide
IVGSQVT

SEQ ID NO: 6 - protein, 12 aa, Homo sapiens
FCDGFYACYMDV

SEQ ID NO: 7 - protein, 12 aa, Artificial Sequence
  Description of Artificial Sequence Synthetic peptide
GWRVSEFGGGSK

SEQ ID NO: 8 - protein, 12 aa, Artificial Sequence
  Description of Artificial Sequence Synthetic peptide
GWRVSEFGGGSK

SEQ ID NO: 9 - protein, 32 aa, Artificial Sequence
  Description of Artificial Sequence Synthetic polypeptide
GWRVSEFKKKKKKKKDDDDFCDGFYACYMDVK

SEQ ID NO: 10 - protein, 25 aa, Artificial Sequence
  Description of Artificial Sequence Synthetic peptide
KKKKKKKKDDDDFCDGFYACYMDVK

SEQ ID NO: 11 - protein, 7 aa, Artificial Sequence
  Description of Artificial Sequence Synthetic peptide
  MOD_RES (2)..(4): Any amino acid
  MOD_RES (6)..(7): Any amino acid
GXXXSXX

SEQ ID NO: 12 - protein, 7 aa, Artificial Sequence
  Description of Artificial Sequence Synthetic peptide
  MOD_RES (7)..(7): F or L
GWRVSEX